SEQUENCE LISTING (77 sequences)

SEQ ID NO: 1 - protein, 793 aa, Homo sapiens
AGWIQAQQLQGDALRQMQVLYGQHFPIEVRHYLAQWIESQPWDAIDLDNPQDRAQATQLLEGLVQELQKKAEHQVGEDGFLLKIKLGHYATQLQKTYDRCPLELVRCIRHILYNEQRLVREANNCSSPAGILVDAMSQKHLQINQTFEELRLVTQDTENELKKLQQTQEYFIIQYQESLRIQAQFAQLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNECSGEILNNCCVMEYHQATGTLSAHFRNMSLKRIKRADRRGAESVTEEKFTVLFESQFSVGSNELVFQVKTLSLPVVVIVHGSQDHNATATVLWDNAFAEPGRVPFAVPDKVLWPQLCEALNMKFKAEVQSNRGLTKENLVFLAQKLFNNSSSHLEDYSGLSVSWSQFNRENLPGWNYTFWQWFDGVMEVLKKHHKPHWNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPVLAKAVDGYVKPQIKQVVPEFVNASADAGGSSATYMDQAPSPAVCPQAPYNMYPQNPDHVLDQDGEFDLDETMDVARHVEELLRRPMDSLDSRLSPPAGLFTSARGSLS

SEQ ID NO: 2 - protein, 787 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 3 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 4 - DNA, 15 bp, Homo sapiens
  source 1..15: mol_type = genomic DNA
cttttccaagaaagc

SEQ ID NO: 5 - DNA, 15 bp, Homo sapiens
  source 1..15: mol_type = genomic DNA
cttttccaagaaagc

SEQ ID NO: 6 - DNA, 24 bp, Homo sapiens
  source 1..24: mol_type = genomic DNA
tcctagttactgtcacactaggaa

SEQ ID NO: 7 - DNA, 24 bp, Homo sapiens
  source 1..24: mol_type = genomic DNA
taaatggcaaaaaaatgcctagaa

SEQ ID NO: 8-9 - skipped (2 entries)

SEQ ID NO: 10 - DNA, 24 bp, Homo sapiens
  source 1..24: mol_type = genomic DNA
ttacaggagtttgggctccttcag

SEQ ID NO: 11 - DNA, 24 bp, Homo sapiens
  source 1..24: mol_type = genomic DNA
tgccaggaaaggccttaccacaag

SEQ ID NO: 12 - DNA, 20 bp, Homo sapiens
  source 1..20: mol_type = genomic DNA
gcattggaatcagacagcac

SEQ ID NO: 13 - DNA, 20 bp, Homo sapiens
  source 1..20: mol_type = genomic DNA
ccacgacgtagtccatgttc

SEQ ID NO: 14 - DNA, 21 bp, Homo sapiens
  source 1..21: mol_type = genomic DNA
attcctgtagatccgagcacc

SEQ ID NO: 15 - DNA, 20 bp, Homo sapiens
  source 1..20: mol_type = genomic DNA
gctcacgtcgctcattttgc

SEQ ID NO: 16 - DNA, 20 bp, Homo sapiens
  source 1..20: mol_type = genomic DNA
gagctgggatgtggtaaagg

SEQ ID NO: 17 - DNA, 20 bp, Homo sapiens
  source 1..20: mol_type = genomic DNA
aaattcagaagatggagcgg

SEQ ID NO: 18 - DNA, 18 bp, Homo sapiens
  source 1..18: mol_type = genomic DNA
tgtccgtcagaacccatg

SEQ ID NO: 19 - DNA, 21 bp, Homo sapiens
  source 1..21: mol_type = genomic DNA
aaagtcgaagttccatcgctc

SEQ ID NO: 20 - DNA, 21 bp, Homo sapiens
  source 1..21: mol_type = genomic DNA
acactaagggccgaagataac

SEQ ID NO: 21 - DNA, 22 bp, Homo sapiens
  source 1..22: mol_type = genomic DNA
acagcatctccaatatggctga

SEQ ID NO: 22 - DNA, 21 bp, Homo sapiens
  source 1..21: mol_type = genomic DNA
gaggttggctctgactgtacc

SEQ ID NO: 23 - DNA, 21 bp, Homo sapiens
  source 1..21: mol_type = genomic DNA
tccgtcccagtagattaccac

SEQ ID NO: 24 - DNA, 20 bp, Homo sapiens
  source 1..20: mol_type = genomic DNA
atgggatgggtgtctccaca

SEQ ID NO: 25 - DNA, 19 bp, Homo sapiens
  source 1..19: mol_type = genomic DNA
ccacgaaggggaacttgtc

SEQ ID NO: 26 - DNA, 23 bp, Homo sapiens
  source 1..23: mol_type = genomic DNA
tatactggctggctagatcactg

SEQ ID NO: 27 - DNA, 23 bp, Homo sapiens
  source 1..23: mol_type = genomic DNA
ggcaaaattggtcccacctataa

SEQ ID NO: 28 - DNA, 20 bp, Homo sapiens
  source 1..20: mol_type = genomic DNA
cagcagcttgacacacggta

SEQ ID NO: 29 - DNA, 22 bp, Homo sapiens
  source 1..22: mol_type = genomic DNA
gcccaatcttgactctcaatcc

SEQ ID NO: 30 - DNA, 20 bp, Homo sapiens
  source 1..20: mol_type = genomic DNA
atgtgaaaccacagatcaag

SEQ ID NO: 31 - DNA, 21 bp, Homo sapiens
  source 1..21: mol_type = genomic DNA
tctgtgggtacatgttatagg

SEQ ID NO: 32 - DNA, 20 bp, Homo sapiens
  source 1..20: mol_type = genomic DNA
atgggactcagtagatcttg

SEQ ID NO: 33 - DNA, 22 bp, Homo sapiens
  source 1..22: mol_type = genomic DNA
cttcagtaaaaacccatcttcc

SEQ ID NO: 34 - DNA, 19 bp, Homo sapiens
  source 1..19: mol_type = genomic DNA
ctgcatctggtcacggtcg

SEQ ID NO: 35 - DNA, 20 bp, Homo sapiens
  source 1..20: mol_type = genomic DNA
cctcgggctcaggatagtct

SEQ ID NO: 36 - DNA, 22 bp, Homo sapiens
  source 1..22: mol_type = genomic DNA
ccccctttacaagcactaatgg

SEQ ID NO: 37 - DNA, 21 bp, Homo sapiens
  source 1..21: mol_type = genomic DNA
ggcagacagtcagaagagctg

SEQ ID NO: 38 - DNA, 21 bp, Homo sapiens
  source 1..21: mol_type = genomic DNA
aaaagacagctacgtgggtga

SEQ ID NO: 39 - DNA, 22 bp, Homo sapiens
  source 1..22: mol_type = genomic DNA
gccatgttctatcgggtacttc

SEQ ID NO: 40 - DNA, 23 bp, Homo sapiens
  source 1..23: mol_type = genomic DNA
tcgtcataatctgtccctacaca

SEQ ID NO: 41 - DNA, 20 bp, Homo sapiens
  source 1..20: mol_type = genomic DNA
cggcttcggctcttagcaaa

SEQ ID NO: 42 - DNA, 20 bp, Homo sapiens
  source 1..20: mol_type = genomic DNA
gtaacccgttgaaccccatt

SEQ ID NO: 43 - DNA, 20 bp, Homo sapiens
  source 1..20: mol_type = genomic DNA
ccatccaatcggtagtagcg

SEQ ID NO: 44 - DNA, 19 bp, Homo sapiens
  source 1..19: mol_type = genomic DNA
cgccaagagactcgtctgg

SEQ ID NO: 45 - DNA, 21 bp, Homo sapiens
  source 1..21: mol_type = genomic DNA
tctttcccaaccgtgaccttc

SEQ ID NO: 46 - DNA, 22 bp, Homo sapiens
  source 1..22: mol_type = genomic DNA
aaattcggtacatcctcgacgg

SEQ ID NO: 47 - DNA, 23 bp, Homo sapiens
  source 1..23: mol_type = genomic DNA
ggaaggttcaggttgttttctgc

SEQ ID NO: 48 - DNA, 22 bp, Homo sapiens
  source 1..22: mol_type = genomic DNA
ggaaactaatctggattcactc

SEQ ID NO: 49 - DNA, 20 bp, Homo sapiens
  source 1..20: mol_type = genomic DNA
catctctagtttcaaccgtc

SEQ ID NO: 50 - DNA, 19 bp, Homo sapiens
  source 1..19: mol_type = genomic DNA
gccaggtgctcaaaggcta

SEQ ID NO: 51 - DNA, 22 bp, Homo sapiens
  source 1..22: mol_type = genomic DNA
tctcgttcagaagtctccagag

SEQ ID NO: 52 - DNA, 20 bp, Homo sapiens
  source 1..20: mol_type = genomic DNA
cccacatgaagcgacttccc

SEQ ID NO: 53 - DNA, 21 bp, Homo sapiens
  source 1..21: mol_type = genomic DNA
caggtccaggagatcgttgaa

SEQ ID NO: 54 - DNA, 22 bp, Homo sapiens
  source 1..22: mol_type = genomic DNA
agtgcagtccaaaatcgagaag

SEQ ID NO: 55 - DNA, 20 bp, Homo sapiens
  source 1..20: mol_type = genomic DNA
cttgctcagaatcacgccat

SEQ ID NO: 56 - DNA, 22 bp, Homo sapiens
  source 1..22: mol_type = genomic DNA
cctctctctaatcagccctctg

SEQ ID NO: 57 - DNA, 21 bp, Homo sapiens
  source 1..21: mol_type = genomic DNA
gaggacctgggagtagatgag

SEQ ID NO: 58 - DNA, 20 bp, Homo sapiens
  source 1..20: mol_type = genomic DNA
ggactgtgacgagttggctg

SEQ ID NO: 59 - DNA, 19 bp, Homo sapiens
  source 1..19: mol_type = genomic DNA
ccgtagaagcgccgatagg

SEQ ID NO: 60 - DNA, 21 bp, Homo sapiens
  source 1..21: mol_type = genomic DNA
caaaaagtcctaacccctgct

SEQ ID NO: 61 - DNA, 19 bp, Homo sapiens
  source 1..19: mol_type = genomic DNA
tcatgtcgtccacctccac

SEQ ID NO: 62 - DNA, 19 bp, Homo sapiens
  source 1..19: mol_type = genomic DNA
acccgctgtcttctagcgt

SEQ ID NO: 63 - DNA, 20 bp, Homo sapiens
  source 1..20: mol_type = genomic DNA
taaattgaagaagaagcgcc

SEQ ID NO: 64 - DNA, 20 bp, Homo sapiens
  source 1..20: mol_type = genomic DNA
tgctccaatgccacagttcc

SEQ ID NO: 65 - DNA, 21 bp, Homo sapiens
  source 1..21: mol_type = genomic DNA
ctgctgaattccattgccaca

SEQ ID NO: 66 - DNA, 21 bp, Homo sapiens
  source 1..21: mol_type = genomic DNA
gtcaacccaaaattggcacca

SEQ ID NO: 67 - DNA, 22 bp, Homo sapiens
  source 1..22: mol_type = genomic DNA
accttgtttcctttcgtcttcg

SEQ ID NO: 68 - DNA, 20 bp, Homo sapiens
  source 1..20: mol_type = genomic DNA
caccaattcctgggaagtct

SEQ ID NO: 69 - DNA, 20 bp, Homo sapiens
  source 1..20: mol_type = genomic DNA
gcagctgttcactttgagga

SEQ ID NO: 70 - DNA, 19 bp, Homo sapiens
  source 1..19: mol_type = genomic DNA
ctgggctacactgagcacc

SEQ ID NO: 71 - DNA, 21 bp, Homo sapiens
  source 1..21: mol_type = genomic DNA
aagtggtcgttgagggcaatg

SEQ ID NO: 72 - DNA, 20 bp, Homo sapiens
  source 1..20: mol_type = genomic DNA
accttacaggagtttgggct

SEQ ID NO: 73 - DNA, 23 bp, Homo sapiens
  source 1..23: mol_type = genomic DNA
ctttcctggcatccagaaggata

SEQ ID NO: 74 - DNA, 19 bp, Homo sapiens
  source 1..19: mol_type = genomic DNA
ccgcccaccccatttactt

SEQ ID NO: 75 - DNA, 23 bp, Homo sapiens
  source 1..23: mol_type = genomic DNA
ggcgaacagaccaattttctagg

SEQ ID NO: 76 - DNA, 25 bp, Homo sapiens
  source 1..25: mol_type = genomic DNA
ggtttttgagggtgagggtgagggt

SEQ ID NO: 77 - DNA, 26 bp, Homo sapiens
  source 1..26: mol_type = genomic DNA
tcccgactatccctatccctatccct

(19) United States
(12) Patent Application Publication
(10) Pub. No.: US 2026/0256761 A1
(43) Pub. Date: Sep. 3, 2026

(54) COMPOSITIONS AND METHODS FOR THE IDENTIFICATION OF INHIBITORS OF AND TREATMENT FOR ECTOPIC CALCIFICATION

(71) Applicant: UNIVERSITY OF PITTSBURGH-OF THE COMMONWEALTH SYSTEM OF HIGHER EDUCATION, Pittsburgh, PA (US)

(72) Inventors: Cynthia L. ST. HILAIRE, Pittsburgh, PA (US); Rolando A. CUEVAS, Pittsburgh, PA (US)

(21) Appl. No.: 18/858,147

(22) PCT Filed: Apr. 6, 2023
(86) PCT No.: PCT/US2023/065430

§ 371 Date: Oct. 18, 2024

Related U.S. Application Data
(60) Provisional application No. 63/331,943, filed on Apr. 18, 2022.

(51) Int. Cl. A61K 31/454; A61F 2/24; A61F 2/82; A61K 31/196; A61K 31/455; A61K 31/661; A61K 45/06; A61P 9/10; G01N 33/68

(52) U.S. Cl. CPC A61K 31/454; A61F 2/24; A61F 2/82; A61K 31/196; A61K 31/455; A61K 31/661; A61K 45/06; A61P 9/10; G01N 33/6893; G01N 2500/02; G01N 2500/20

(57) ABSTRACT

The present disclosure relates to treating ectopic calcification using Signal transducer arid activator of transcription 5 (STAT5) and/or Telomerase reverse transcriptase (TERT) inhibitors, methods for identifying one or more small molecule inhibitors to reduce calcification of a circulatory system member or a device, and devices comprising a STAT5 and/or a TERT inhibitor.

### RELATED APPLICATION

This PCT application claims priority to, and the benefit of, U.S. Provisional Patent Application No. 63/331,943, filed Apr. 18, 2022, which is incorporated by reference herein in its entirety.

### STATEMENT REGARDING FEDERALLY SPONSORED RESEARCH

This invention was made with Government support under HL142932 awarded by the National Institutes of Health. The U.S. Government has certain rights in this invention.

### REFERNCE TO SEQUENCE LISTING

The sequence listing submitted on Apr. 6, 2023, as an .XML entitled “10504-079WO1_ST26.xml” created on Apr. 6, 2023, and having a file size of 69,855 bytes is hereby incorporated by reference pursuant to 37 C.F.R. § 1.52(e) (5).

### BACKGROUND OF THE INVENTION

### 1) Field of the Invention

The present disclosure relates to treating ectopic calcification using STAT5 and/or TERT inhibitors including methods for screening for STAT5 and/or TERT inhibitors.

### 2) Description of Related Art

Ectopic cardiovascular calcification is a complex and dynamic process, with many initiating stressors and intracellular signaling pathways contributing to this pathology. Microcalcifications in atherosclerotic plaque distorts mechanical load bearing which contributes to plaque rupture. Medial arterial calcification in the lower extremity arteries is a leading cause of chronic and acute limb ischemia in peripheral artery disease, and calcification of the aortic valve incurs a higher risk of stroke and ultimately leads to heart failure. The numbers of individuals with ectopic cardiovascular calcification are huge: in 2015 over 7.4 million individuals died of coronary heart disease, of which atherosclerotic calcification is a biomarker for and a contributor to plaque rupture. Calcific aortic valve disease (CAVD) encompasses a spectrum of pathological remodeling of the aortic valve leaflet. Aortic sclerosis is defined as the thickening and stiffening of the valve leaflets and is present in approximately 34% of individuals ≥65 years of age. Peripheral artery disease (PAD) exhibits atherosclerotic calcification as well as medial layer calcification and has been diagnosed in over 230 million people worldwide and is estimated to be found in 10-20% of individuals over the age of 60. Importantly, PAD is commonly found alongside comorbidities such as diabetes, hypertension, and chronic kidney disease (CKD), and with the presence of risk factors such as smoking, obesity, and hyperlipidemia.

While statins and anticoagulant therapies target lipids and platelet-mediated complications of vascular remodeling, there is currently no therapy that can prevent, halt, or reverse ectopic cardiovascular calcification. At present, the only therapy for CAVD is aortic valve replacement. While metal valves are durable, they require life-long anticoagulant therapy. Bioprosthetic valves are now more commonly used as they can be implanted via catheter, however their lifespan is only approximately 10 years.

Given the limited availability of therapies and treatments for cardiovascular calcification, there is a need, therefore, to develop compositions and methods for treating calcification.

### SUMMARY

The present disclosure provides methods of treating and/or preventing calcification in cardiovascular related systems, tissues, and devices.

In one aspect, disclosed herein is a method of treating calcification of a circulatory system member in a subject comprising administering to the subject a pharmaceutically effective amount of a STAT5 inhibitor and/or a TERT inhibitor. In some embodiments, the circulatory system member is a heart valve. In some embodiments, the circulatory system member is a heart tissue. In some embodiments, the circulatory system member is a heart vein or a heart artery.

In some embodiments, the subject has a calcific aortic heart disease, atherosclerotic plaques in an artery, or medial arterial calcification.

In some embodiments, the STAT5 inhibitor is administered to a vein of the subject. In some embodiments, the STAT5 inhibitor is administered to a vein of the subject selected from the group consisting of a subclavian vein, a thoracic internal vein, an axillary vein, a cephalic vein, a brachial vein, a basilic vein, an intercostal vein, a median cubital vein, a cephalic vein, an ulnar vein, a median antebrachial vein, and a palmar vein.

In some embodiments, the STAT5 inhibitor and/or the TERT inhibitor reduces osteogenic differentiation of a cell. In some embodiments, the STAT5 inhibitor and/or the TERT inhibitor reduces osteogenic differentiation of a valve interstitial cell, valve endothelial cell, vascular endothelial cell, vascular smooth muscle cell, adventitial fibroblast, cardiomyocyte, cardiac fibroblast, circulating mesenchymal stem cell, and/or a resident mesenchymal stem cell.

In one aspect, disclosed herein is a method of reducing calcification of a device within a subject's circulatory system comprising applying to the device a pharmaceutically effective amount of a STAT5 inhibitor and/or a TERT inhibitor.

In some embodiments, the device is a bioprosthetic heart valve. In some embodiments, the device is a stent.

In some embodiments, the STAT5 inhibitor and/or the TERT inhibitor is applied to the device prior to placement in the subject. In some embodiments, the STAT5 inhibitor is selected from the group consisting of a StafiA-1, a StafiB-1, a pimozide, a nicotinoyl hydrazine, a BP-1-108, an AC-4-130, and a SF-1-088.

In some embodiments, the STAT5 inhibitor is a StafiA-1. In some embodiments, the STAT5 inhibitor is a StafiB-1. In some embodiments, the STAT5 inhibitor is a pimozide. In some embodiments, the STAT5 is a STAT5A. In some embodiments, the STAT5 is STAT5B.

In some embodiments, the STAT5 inhibitor is administered or applied.

In one aspect, disclosed herein is a method of screening one or more small molecule inhibitors to inhibit or reduce calcification, the method comprising introducing a STAT5 polypeptide and a TERT polypeptide in the presence of the one or more small molecule inhibitors, identifying the one or more small molecule inhibitors that reduce or prevent interaction between the STAT5 and TERT polypeptides, and wherein the one or more small molecule inhibitors reduce calcification of a circulatory system member or calcification of a device within a subject's circulatory system.

In some embodiments, the method includes detecting and visualizing an interaction between the STAT5 and TERT proteins, wherein the small molecule inhibitor prevents or reduces the interaction between STAT5 and TERT.

In some embodiments, the small molecule inhibitor is a peptide, polypeptide, nucleic acid, composition, or compound.

In some embodiments, the method detects a visual signal when STAT5 and TERT interact. In some embodiments, the visual signal is decreased in the presence of the small molecule inhibitor.

In some embodiments, the small molecule inhibitor treats or reduces calcification of a circulatory member or calcification of a device within a subject's circulatory system.

In some embodiments, the circulatory system member is a heart valve. In some embodiments, the circulatory system member is a heart tissue. In some embodiments, the circulatory system member is a heart vein or a heart artery. In some embodiments, the device comprises a bioprosthetic heart valve or a stent.

In some embodiments, the subject is an animal or a human.

### BRIEF DESCRIPTION OF FIGURES

The accompanying figures, which are incorporated in and constitute a part of this specification, illustrate several aspects described below.

FIG. 1(A-E) shows TERT is upregulated in CAVD valve tissue. FIG. 1A shows the anatomic specimen of control (left) and CAVD (right) human valves. FIG. 1B shows TERT mRNA quantification in control and CAVD valve tissues. n=8 control, n=9 CAVD. FIG. 1C shows representative serial sections of Von Kossa (dark precipitation, top panels) and TERT immunofluorescent staining (bottom panels) in control and CAVD valve tissues. Scalebar 100 μm. Quantification of TERT-positive cells on the leaflet subject is showed on the right graph. n=9 control, n=9 CAVD. FIG. D shows the telomere length measurements in valve tissues. n=16, control n=10 CAVD. FIG. E shows the age of the patients utilized in this study. n=25 control, n=16 CAVD. Data represents means±SD and P were calculated by Mann-Whitney U test. P values are indicated on each graph. ns=not significant.

FIG. 2(A-E) shows AVICs isolated from CAVD patients show elevated TERT expression at baseline. FIG. 2A shows representative images showing cell morphology of control (left) and CAVD (right) AVICs. Scalebar 50 μm. FIG. 2B shows proliferation of control and CAVD AVICs at baseline. n=3 control, n=3 CAVD; data represents means±SD. FIG. 2C shows the relative migration distances of control and CAVD AVICs. n=10 control, n=10 CAVD; means±SD. FIG. 2D shows representative western blot staining of control and CAVD AVICs at baseline. Quantification of protein levels is shown on the right panels. n=4, control n=4 CAVD; means±SD. FIG. 2E Telomere length measurements in AVICs. n=8, control n=5 CAVD; means±SD. All graphs and P were calculated by Mann-Whitney U test. P values are indicated on each graph. ns=not significant.

FIG. 3(A-D) shows osteogenic stimulation induces expression of TERT in AVICs. FIG. 3A shows representative images of AVICs under normal growth conditions (no treatment, NT) or stimulated with osteogenic media (OST) for 14 days. Calcium deposition was visualized by Alizarin Red staining. Scalebar 50 μm. Quantification of calcification is represented as average absorbance on right graph. n=4 control, n=4 CAVD. FIG. 3B shows representative immunofluorescent staining images of TERT in control and CAVD AVICs stimulated with NT or OST for 14 days. Calcium deposition was visualized by OsteoImage staining. Scalebar 100 μm. FIG. 3C shows representative western blot staining of samples from control and CAVD AVICs after 14 days of NT or OST. Quantification of protein levels is shown on right graphs. n=4 control, n=4 CAVD. FIG. 3D shows the expression profile control AVICs after 14 days of NT and OST treatment. n=4 NT, n=4 OST. Data is represented as means±SD. P were calculated by Mann-Whitney U test. *P=0.0286.

FIG. 4(A-D) shows TERT is required for osteogenic transition and calcification. FIG. 4A shows representative western blot staining of human AVICs transduced with lentivirus containing either short hairpin scramble (shControl-GFP) or short hairpin TERT (shTERT-GFP) followed by 7 days of osteogenic stimulation. FIG. 4B shows Human CASMCs transduced with lentivirus containing either shControl-GFP or shTERT-GFP and stimulated with osteogenic media for up to 21 days. Calcium deposition was visualized by Alizarin Red staining. Quantification of calcification is shown as average absorbance on right graph. n=4; means±SD; two-way ANOVA. FIG. 4C shows representative western blot of human MSCs transduced with lentivirus containing either shControl-GFP or shTERT-GFP followed by 7 days of osteogenic stimulation. FIG. 4D shows human CASMCs transduced with lentivirus containing either shControl-GFP or shTERT-GFP and stimulated with osteogenic media for up to 18 days. Quantification of calcification is shown as average absorbance on right graph. n=4; means±SD; two-way ANOVA.

FIG. 5(A-B) shows TERT is required for osteogenic transition and calcification of mice cells. FIG. 5A shows representative images of mAVICs isolated from either wild-type (WT) or F1 Tert knockout (Tert^{-/-}) mice and stimulated with osteogenic media for 21 days. Scalebar 100 μm. Quantification of calcification shown on right graph. FIG. 5B shows representative images of mBMMSCs isolated from either wild-type (WT) or Tert knockout (Tert^{-/-}) mice and stimulated with osteogenic media for 21 days. Scalebar 100 μm. Quantification of calcification is represented as average absorbance on the right graph. All graphs are n=4 WT; n=4 Tert^{-/-}. Data represents means±SD. P were calculated by Mann-Whitney U test and indicated in the graphs.

FIG. 6(A-E) shows TERT and STAT5 interact to upregulate RUNX2 expression in AVICs. FIG. 6A shows the diagram of proximity ligation assay (PLA, left panel) Representative images of TERT/STAT5 complex (red foci) in CAVD AVICs cultured for 21 days in osteogenic medium and detected by PLA. Scalebar 100 μm (middle panel). Quantification of PLA signal is shown on the right graphs. n=4. FIG. 6B shows the logo analysis depicting the consensus binding site for tetrameric STAT5 (top panel). Diagram depicting STAT5 and TERT binding RUNX2 promoter and the positions of the predicted STAT5 binding sites (bottom panel). FIG. 6C shows STAT5 (left graphs) and TERT (right graphs) enrichment on the RUNX2 promoter after 14 days of osteogenic stimulation and quantified by qPCR n=4, control. FIG. 6D shows representative images of AVICs treated with 10 uM of the STAT5 inhibitors StafiA-1 or StafiB-1 during 28 days of osteogenic stimulation. Scalebar 400 μm n=8; means±SD. Quantification of calcification is shown on the right graph. Data is represented as means±SD. P were calculated by Kruskal-Wallis H test with Dunn pairwise comparison post hoc test. Scalebar 400 um. FIG. 6E shows that human coronary smooth muscle cells were seeded at a density of 250,000 cells per 9.5 cm². When the cells reached confluence, media was replaced with osteogenic media (Minimum Essential Medium alpha with nucleosides, 10% FBS, 1×P/S, 10 mM glycerol phosphate, 50 μM ascorbic acid 2-phosphate, and 100 nM dexamethasone) for 21 days. No treatment media consisted of Minimum Essential Medium alpha with nucleosides, 10% FBS, 1×P/S. Media was replaced every four days. StafiA-1, 10 μM; StafiB-1 10 μM.

FIG. 7(A-B) shows TERT and STAT5 are upregulated and localize in CAVD tissue. FIG. 7A shows representative serial sections of Von Kossa (dark precipitation, top panels) and TERT and STAT5 immunofluorescent staining (bottom panels) in control and CAVD valve tissues. Scalebar 100 μm. FIG. 7B shows the quantification of STAT5-positive cells on the leaflet subject (left panel). Quantification of STAT5-TERT-positive cells on the leaflet subject (right panel). Data is presented as means±SD, n=5 control, n=6 CAVD. P were calculated by Mann-Whitney U test and indicated in each graph.

FIG. 8 shows TERT/STAT5 promotes osteogenic reprogramming. Inflammation promotes the upregulation and interaction of TERT and STAT5. Together, the complex translocates into the nucleus where TERT facilitates STAT5 binding to the promoter of RUNX2 and initiating the osteogenic reprogramming of AVICs in the early stages of CAVD pathogenesis.

FIG. 9(A-B) shows that TERT interacts with STAT5 and the beta-catenin-BRG1 complex. FIG. 9A shows the coimmunoprecipitation of endogenous TERT and BRG1 from hVICs growing in control media (NT) or osteogenic differentiation media (OST) for ten days. FIG. 9B shows the coimmunoprecipitation of endogenous STAT5 and TERT, beta-catenin, and BRG1 from hVICs from hVICs growing in NT or OST media for ten days. These data show that TERT and STAT5 physically interact and form a higher hierarchical complex with the beta-catenin-BRG1 complex during the osteogenic differentiation of hVICs.

FIG. 10(A-B) shows that TERT and STAT5 colocalize to the remodeling machinery in CAVD tissue. FIG. 10A shows the immunofluorescent staining of calcified CAVD tissue showing expression of TERT and the chromatin remodeling molecule BRG1. Scale bar 200 μm. Colocalization of TERT and BRG1 is shown in insets with higher magnification (scale bar of insets indicates 25 μm). FIG. 10B shows the immunofluorescent staining of calcified CAVD tissue showing expression of beta-catenin and STAT5. Scale bar 200 μm. Colocalization of STAT5 and beta-catenin is shown in insets with higher magnification (scale bar of insets indicates 25 μm). These data show that TERT and STAT5 work in concert with the chromatin remodeling machinery to regulate transcription by altering the chromatin structure around their cognate targets.

FIG. 11(A-E) shows the characterization of human CAVD valves and correlation of TERT expression with age. FIG. 11A shows representative section of Von Kossa staining in control and valve CAVD leaflet tissues. Scalebar 1 mm. FIG. 11B shows the representative serial sections of RUNX immunofluorescent staining (top panels) and OPN (bottom panels) in control and CAVD valve tissues. Scalebar 100 um. Quantification of RUNX-positive cell and OPN-positive cells on the leaflet subject is shown on the right graphs. n=5 control, n=6 CAVD. FIG. 11C shows correlation between age of donor and TERT expression, n=8 control, n=9 CAVD; Pearson correlation test. FIG. 11D shows the expression profile in the subject leaflets n=7 control, n=7 CAVD. FIG. 11E shows representative images of PCNA immunofluorescent staining (top panels) and H2AX (bottom panels) in control and CAVD valve tissues. Scalebar 100 um. Quantification of PCNA-positive cells and H2AX-positive cells on the leaflet subject is shown on the right graphs n=4 control, n=3 CAVD. Data represents means±SD. P were calculated by Mann-Whitney U test (B and E) and two-way ANOVA with Sidak post hoc test (D). *P<0.05, **<0.005, and ns=not significant.

FIG. 12(A-C) shows the AVIC migration properties and expression of activation and osteogenic markers at baseline. FIG. 12A shows representative images of a scratch assay in control (top panels) and CAVD (bottom panels) AVICs Time points is indicated on top. Scalebar 1 mm. n=10 control, n=10 CAVD. FIG. 12B shows representative images of αSMA and CNN (top panels) and SM22 (bottom panels) in control and CAVD AVICs. Scalebar 50 um. Signal quantification of αSMA, CNN, and SM22 are shown on the right panels. n=4 control, n=4 CAVD. Means±SD. FIG. 12C shows representative images of TNAP and f-Actin signal in control and CAVD AVICs. Scalebar 50 um. Signal quantification of TNAP and f-Actin is shown on the right panels. n=4 control, n=4 CAVD. Data represent means±SD and P were calculated by Mann-Whitney U test with * P<0.05 and ns=not significant.

FIG. 13(A-C) shows the characterization of human AVICs during osteogenic differentiation and inhibition of telomere-extending activity of TERT by BiBR1532. FIG. 13A shows the cell counting of control and CAVD AVIC lines treated with NT or OST media for 14 days. n=4 control, n=4 CAVD. FIG. 13B shows representative images of senescence-associated β-galactosidase activity staining of AVICs stimulated with OST media for 28 days. Blue cells were considered to be positive for senescence. Quantification of senescence is shown on the right graph. n=4 control, n=4 CAVD. FIG. 13C shows the AVICs treated with BiBR1532 and stimulated with OST for 28 days. Scalebar 50 um. Quantification of calcification is shown on the right graph. n=4. Data represents means±SD and P were calculated by Kruskal-Wallis H test with Dunn post hoc test. ns=not significant.

FIG. 14(A-D) shows TERT and RUNX2 expression during osteogenic differentiation of human mesenchymal stem cells. FIG. 14A shows representative images of human MSCs stimulated with OST for 14 days. Calcium deposition was visualized by Alizarin Red staining. Quantification of calcification is shown on the right graph. n=3. FIG. 14B shows the western blot staining of samples from MSCs collected during the 14 days of OST treatment. FIG. 14C shows TERT and RUNX2 expression profile of differentiating MSCs stimulated for 14 days with OST media. n=3. FIG. 14D shows representative TERT immunofluorescent staining images of human MSCs stimulated with osteogenic media for 14 days. Data is represented as means±SD. P were calculated by Kruskal-Wallis H test with Dunn pairwise comparison post hoc test (A and C).

FIG. 15(A-B) shows senescence is not operative in mice Tert^{-/-} cells. FIG. 15A shows representative images of a senescence-associated β-galactosidase (SA-β-gal) staining of mVICs after 21 days of osteogenic stimulation. Blue cells were considered to be positive for senescence. Quantification of senescence cells is shown on the right graph. FIG. 15B shows representative images of SA-β-gal staining of mBMMSCs after 21 days of osteogenic stimulation. Blue cells were considered to be positive for senescence. Quantification of senescence is shows on the right graph. Both figures, n=5 WT; n=3 Tert. Bar represents 400 um. Data represents means±SD and P were calculated by Mann-Whitney U test. ns=not significant.

FIG. 16(A-F) shows that STAT5 is required for the calcification of human AVICs. FIG. 16A shows the representative western blot staining of samples from CAVD and control AVICs after 14 days of osteogenic stimulation. Quantification of protein levels. n=4 control, n=4 CAVD; means±SD; two-way ANOVA. FIG. 16B shows representative immunofluorescence image of STAT5 subcellular redistribution after osteogenic 14 days of osteogenic stimulation. FIG. 16C shows representative images of TERT/STAT5 complex (red foci) in control AVICs and detected by proximity ligation assay (PLA). Scalebar 100 um. Quantification of PLA is shown on the right graph. n=4. FIG. 16D shows representative images of CASMC treated with 10 uM of the STAT5 inhibitors. STAT5 inhibitors were added to the media at the following concentrations: StafiA-10 uM; StafiB-1 10 uM. n=4 for all conditions; data is represented as means±SD. P were calculated by Kruskal-Wallis H test with Dunn pairwise comparison post hoc test. Scalebar 400 um. FIG. 16E shows the representative immunofluorescence images of AVICs after 14 days of LPS treatment or osteogenic stimulation. Scalebar 50 um. FIG. 16F shows representative western blot staining of CAVD AVICs after 14 days of osteogenic stimulation.

FIG. 17 shows human mesenchymal stem cells seeded at a density of 250,000 cells per 9.5 cm². When cells reached confluence, media was replaced with osteogenic media (Minimum Essential Medium alpha with nucleosides, 10% FBS 1×P/S, 10 mM glycerol phosphate, 50 uM ascorbic acid 2-phosphate, and 100 nM dexamethasone) for 21 days. No treatment media consisted of Minimum Essential Medium alpha with nucleosides, 10% FBS, 1×P/S. Media was replaced every four days. STAT5 inhibitors were added to the media at the following concentrations: StafiA-10 uM; StafiB-1 10 uM. n=4 for all conditions; data is represented as means±SD. P were calculated by Kruskal-Wallis H test with Dunn pairwise comparison post hoc test. Scalebar 400 μm.

FIG. 18(A-B) shows the TERT and STAT5 colocalize in prosthetic valve tissue. FIG. 18A shows the immunofluorescent stain of an explanted aortic valve replacement showing strong expression and colocalization of TERT and STAT5. Scale bar 200 μm. Colocalization of TERT and STAT5 is shown in insets with higher magnification. FIG. 18B shows the confocal imaging shows that TERT and STAT5 colocalize in the nucleus of the cells in aortic valve replacement tissue. Scale bar 10 μm. These data show that TERT and STAT5 are also operative in the calcification observed on prosthetic valve tissue.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

The following definitions are provided for the full understanding of terms used in this specification. Unless defined otherwise, all technical and scientific terms used herein have the same meaning as commonly understood to one of ordinary skill in the art to which this disclosure belongs.

As used in this disclosure and in the appended claims, the singular forms “a”, “an”, “the”, include plural referents unless the context clearly dictates otherwise. For example, the term “a cell” includes a plurality of cells, including mixtures thereof.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques. In some embodiments, the administration is intravenous.

The word “calcification” refers herein to the deposition of or conversion of calcium and phosphate into hydroxyapatite, or other mineral compound formulations that precipitate and accumulate in the cardiovascular system. In some embodiments, calcification of a tissue results in the stiffening and/or hardening of the tissue.

The disease, “calcific aortic valve disease” or “CAVD,” refers herein to a condition involving calcification of the aortic valve. CAVD can range from mild aortic valve thickening without obstruction of blood flow (aortic sclerosis) to severe calcification with impaired leaflet motion (aortic stenosis). In some embodiments, CAVD can be detected using one or more of an echocardiogram, electrocardiogram, chest x-ray, cardiac MRI, cardiac computerized tomography (CT) scan, and cardiac catheterization.

As used herein, a “circulatory system member” refers to the heart, blood vessels, and all parts of and cell types in the heart and blood vessels. “Blood vessels” include arteries, arterioles, veins, venules, and capillaries. In some embodiments, the circulatory system member is a heart valve. In some embodiments, the circulatory system member is a myocardium tissue, an endocardium tissue or an epicardium tissue. In some embodiments, the circulatory system member is a valve interstitial cell, valve endothelial cell, vascular endothelial cell, vascular smooth muscle cell, adventitial fibroblast, cardiomyocyte, cardiac fibroblast, circulating mesenchymal stem cell, or a resident mesenchymal stem cells.

A “composition” is intended to include a combination of active agent and another compound or composition, inert (for example, a detectable agent or label) or active, such as an adjuvant.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

A “control” is an alternative subject or sample used in an experiment for comparison purpose. A control can be “positive” or “negative.”

The term “identity” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. Neither N- nor C-terminal extensions nor insertions shall be construed as reducing identity. A polynucleotide or polynucleotide region (or a polypeptide or polypeptide region) that has a certain percentage (for example, 80%, 85%, 90%, or 95%) of “sequence identity” to another sequence means that, when aligned over their full lengths, that percentage of bases (or amino acids) are the same in comparing the two sequences. This alignment and the percent sequence identity can be determined using software programs known in the art. In one embodiment, default parameters are used for alignment. In one embodiment a BLAST program is used with default parameters. In one embodiment, BLAST programs BLASTN and BLASTP are used with the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. In some embodiments, the comparison is made over the full length of the compared sequences.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “osteogenic differentiation” is the process in which a cell transitions from its native state and acquires functions that ultimately allow it to produce a calcified matrix composition.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically effective amount”, “therapeutically effective amount” or “therapeutically effective dose” refer to the amount of a compound such as a TERT inhibitor and/or a STAT5 inhibitor or a composition that blocks the interaction or function of these proteins together that will elicit the biological or medical response of a tissue, system, animal, or human that is being sought by the researcher, veterinarian, medical doctor, or other clinician. In some embodiments, a desired response is a decrease in calcification or a halt in the progression of calcification. In some instances, a desired biological or medical response is achieved following administration of multiple dosages of the composition to the subject over a period of days, weeks, or years. The terms “pharmaceutically effective amount”, “therapeutically effective amount” or “therapeutically effective dose” include that amount of a compound such as a TERT inhibitor and/or a STAT5 inhibitor and/or a composition that blocks the interaction or function of these proteins together that, when administered, is sufficient to prevent development of, or alleviate to some extent, one or more of the symptoms of the condition or disorder being treated. The therapeutically effective amount will vary depending on the compound such as a TERT inhibitor and/or a STAT5 inhibitor and/or a composition that blocks the interaction or function of these proteins together, the disorder or conditions and its severity, the route of administration, time of administration, rate of excretion, drug combination, judgment of the treating physician, dosage form, and the age, weight, general health, sex and/or diet of the subject to be treated. In the context of the present method, a pharmaceutically or therapeutically effective amount or dose of a TERT inhibitor and/or a STAT5 inhibitor includes an amount that is sufficient to decrease calcification of a cardiovascular tissue, including a heart valve.

The term “promoter” refers to a sequence of DNA to which proteins binds to initiate transcription of a single RNA transcript from the DNA downstream of the promoter.

As used herein, “reduce” means to decrease by a statistically significant amount. In some embodiments, the reduction is about 10%, 20%, 30%, 40%, 50%, 60%, 70, 80%, or 90%.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the invention may be applied preventively, prophylactically, palliatively or remedially. Treatments are administered to a subject prior to onset (e.g., before obvious signs of calcification), during early onset (e.g., upon initial signs and symptoms of calcification), or after an established development of calcification. Prophylactic administration can occur for several days to years prior to the manifestation of symptoms of calcification.

In some instances, the terms “treat”, “treating”, “treatment” and grammatical variations thereof, include partially or completely reducing the amount (area, volume, or density) or severity of calcification, reducing the rupture of plaques, and/or reducing the amount or severity of calcific aortic valve disease (CAVD), atherosclerosis/atherosclerotic plaques, atherosclerotic peripheral artery disease, or peripheral artery disease with medial arterial calcification as compared with prior to treatment of the subject or as compared with the incidence of such symptom in a general or study population. In some embodiments, atherosclerotic plaques can be detected using cardiac magnetic resonance or carotid ultrasound. In some embodiments, peripheral artery disease can be detected using an Ankle-brachial index.

### Chemical Terminology

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄)alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenyl alcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄)alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, acetal, alkyl, alkoxy, alkenyl, alkynyl, aryl, heteroaryl, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible, and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄)alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, acetal, alkyl, alkoxy, alkenyl, alkynyl, aryl, heteroaryl, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, acetal, alkyl, alkoxy, alkenyl, alkynyl, aryl, heteroaryl, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, acetal, alkyl, alkoxy, alkenyl, alkynyl, aryl, heteroaryl, acyl, aldehyde, amino, cyano, carboxylic acid, carbonate ester, carbamate ester, ester, ether, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, acetal, alkyl, alkoxy, alkenyl, alkynyl, aryl, heteroaryl, acyl, aldehyde, amino, cyano, carboxylic acid, carbonate ester, carbamate ester, ester, ether, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄)alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O) H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

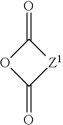

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

A “carbonate ester” group as used herein is represented by the formula Z¹OC(O)OZ².

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

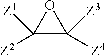

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

The term “Fmoc” as used herein refers to a fluorenylmethyloxycarbonyl group.

The term “Boc” as used herein refers to a t-butyloxycarbonyl group.

The term “Cbz” as used herein refers to a benzyloxycarbonyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

The term “olefinically unsaturated group” or “ethylenically unsaturated group” is employed herein in a broad sense and is intended to encompass any groups containing a carbon-carbon double bonded group (>C═C< group). Exemplary ethylenically unsaturated groups include, but are not limited to, (meth)acrylate, (meth)acrylamide, (meth)acryloyl, allyl, vinyl, styrenyl, or other >C═C< containing groups.

“Polymer” means a material formed by polymerizing one or more monomers.

The term “(co)polymer” includes homopolymers, copolymers, or mixtures thereof.

The term “(meth)acryl . . . ” includes “acryl . . . ,” “methacryl . . . ,” or mixtures thereof.

“Molecular weight” of a polymeric material (including monomeric or macro-monomeric materials), as used herein, refers to the number-average molecular weight as measured by ¹H NMR spectroscopy unless otherwise specifically noted or unless testing conditions indicate otherwise.

### Methods of Treating or Reducing Calcification

In one aspect, disclosed herein is a method of treating calcification of a circulatory system member in a subject comprising administering to the subject a pharmaceutically effective amount of a STAT5 inhibitor and/or a TERT inhibitor and/or a composition that blocks the interaction or function of these proteins together. In some embodiments, a STAT5 inhibitor is administered without a TERT inhibitor.

In some embodiments, the circulatory system member is a heart valve. In some embodiments, the circulatory system member is a heart tissue. In some embodiments, the circulatory system member is a heart vein or a heart artery. In some embodiments, the circulatory system member is a vein or an artery in any area of the body.

In some embodiments, the subject has a calcific aortic heart disease, atherosclerotic plaques in an artery, or medial arterial calcification.

In some embodiments, the STAT5 inhibitor is administered to a vein of the subject. In some embodiments, the STAT5 inhibitor is administered to a vein of the subject selected from the group consisting of a subclavian vein, a thoracic internal vein, an axillary vein, a cephalic vein, a brachial vein, a basilic vein, an intercostal vein, a median cubital vein, a cephalic vein, an ulnar vein, a median antebrachial vein, and a palmar vein.

In some embodiments, the STAT5 inhibitor and/or the TERT inhibitor reduces osteogenic differentiation of a cell. In some embodiments, the STAT5 inhibitor and/or the TERT inhibitor reduces osteogenic differentiation of a valve interstitial cell, valve endothelial cell, vascular endothelial cell, vascular smooth muscle cell, adventitial fibroblast, cardiomyocyte, cardiac fibroblast, circulating mesenchymal stem cell, and/or a resident mesenchymal stem cell.

In one aspect, disclosed herein is a method of reducing calcification of a device within a subject's circulatory system comprising applying to the device a pharmaceutically effective amount of a STAT5 inhibitor and/or a TERT inhibitor and/or a composition that blocks the interaction or function of these proteins together. In some embodiments, the device is a bioprosthetic heart valve. In some embodiments, the device is a stent. In some embodiments the device is a vascular graft. In some embodiments, the application of a STAT5 inhibitor and/or a TERT inhibitor is made prior to the device's placement in the circulatory system member of the subject.

The STAT5 inhibitor and/or a TERT inhibitor applied to the device can be any as described herein. In some embodiments, the STAT5 inhibitor comprises a compound of Formula I, or a pharmaceutically acceptable salt or derivative thereof. In some embodiments, the STAT5 inhibitor comprises a compound of Formula IA, or a pharmaceutically acceptable salt or derivative thereof. In some embodiments, the STAT5 inhibitor comprises a StafiA-1, a StafiB-1, or a derivative thereof. In some embodiments the STAT5 inhibitor comprises a StafiA-1. In some embodiments, the STAT5 inhibitor comprises a StafiB-1. In some embodiments, the STAT5 inhibitor is selected from the group consisting of a StafiA-1, a StafiB-1, a pimozide, a nicotinoyl hydrazine, a BP-1-108, an AC-4-130, and a SF-1-088. In some embodiments, the STAT5 inhibitor comprises a pimozide. In some embodiments, the STAT5 is a STAT5A. In some embodiments, the STAT5 is a STAT5B.

A “STAT5 inhibitor” refers herein to a composition that reduces STAT5 activity, or STAT5 interaction or binding with TERT, reduces STAT5 activation of TERT, and/or reduces STAT5 binding and/or activation of a RUNX2 gene or promoter. Several non-limiting examples of STAT5 inhibitors are provided below.

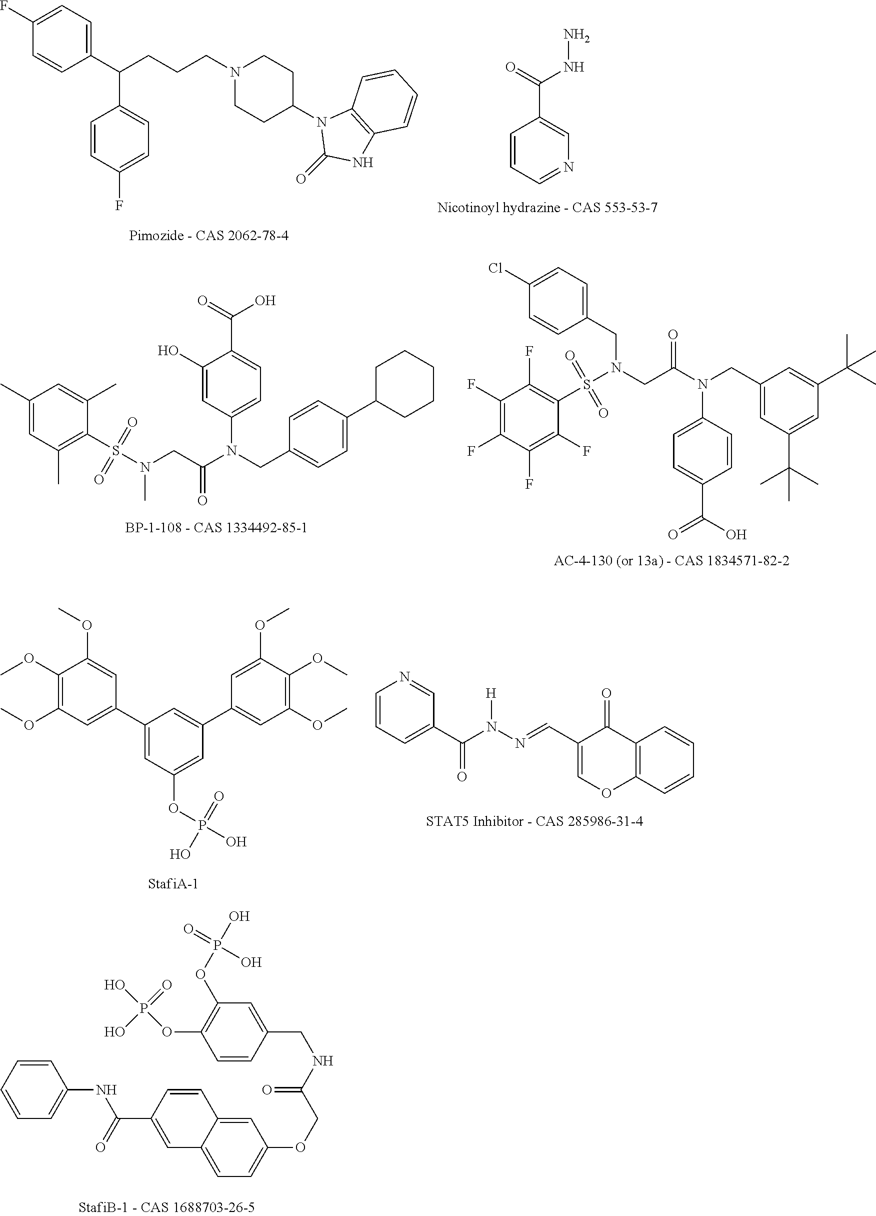

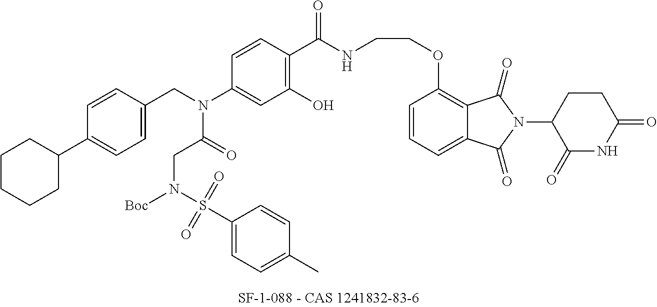 [SMILES: Cc1ccc(S(=O)(=O)N(CC(=O)N(Cc2ccc(C3CCCCC3)cc2)c2ccc(C(=O)NCCOc3cccc4c3C(=O)N(C3CCC(=O)NC3=O)C4=O)c(O)c2)C(=O)OC(C)(C)C)cc1]

In some embodiments, the STAT5 inhibitor and/or the TERT inhibitor is applied to the device prior to placement in the subject. In some embodiments, the STAT5 inhibitor is selected from the group consisting of a StafiA-1, a StafiB-1, a pimozide, a nicotinoyl hydrazine, a BP-1-108, an AC-4-130, and a SF-1-088.
In some embodiments, the STAT5 inhibitor comprises a compound of Formula I, or a pharmaceutically acceptable salt or derivative thereof:
wherein

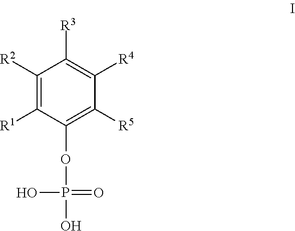

R¹, R³, and R⁵ are independently H, OH, halogen, cyano, nitro, sulfonyl, phosphonyl, substituted or unsubstituted C₁-C₄ alkyl, or NR^{x}R^{y};
R² and R⁴ are independently H, OH, halogen, cyano, nitro, sulfonyl, phosphonyl, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y};
or wherein, as valence permits, R¹ and R², R² and R³, R³ and R⁴, or R⁴ and R⁵, together with the atoms to which they are attached, form a 3-10 membered substituted or unsubstituted cyclic moiety optionally including from 1 to 3 heteroatoms; and
R^{x} and R^{y} are independently selected from H, OH, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula I, R¹, R³, and R⁵ are independently H, OH, halogen, sulfonyl, phosphonyl, substituted or unsubstituted C₁-C₄ alkyl, or NR^{x}R^{y}, and R^{x} and R^{y} are independently selected from H, OH, or substituted or unsubstituted C₁-C₄ alkyl.

In some examples of Formula I, R¹ and R³ are both hydrogen, such that the STAT5 inhibitor comprises a compound of Formula IA:

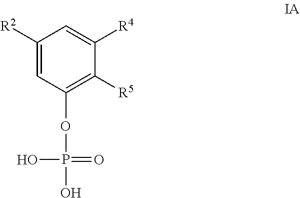

or a pharmaceutically acceptable salt or derivative thereof.

In some examples of Formula I or Formula IA, R⁵ is hydrogen or phosphonyl. In some examples of Formula I or Formula IA, R⁵ is hydrogen. In some examples of Formula I or Formula IA, R⁵ is phosphonyl.

In some examples of Formula I or Formula IA, R² and R⁴ are independently H, OH, halogen, cyano, nitro, sulfonyl, phosphonyl, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}.

In some examples of Formula I or Formula IA, R² and R⁴ are different.

In some examples of Formula I or Formula IA, R⁴ is H, OH, halogen, sulfonyl, phosphonyl, substituted or unsubstituted C₁-C₄ alkyl, or NR^{x}R^{y}, and R^{x} and R^{y} are independently selected from H, OH, or substituted or unsubstituted C₁-C₄ alkyl. In some examples of Formula I or Formula IA, R⁴ is hydrogen.

In some examples of Formula I or Formula IA, R² is substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}. In some examples of Formula I or Formula IA, R² is substituted or unsubstituted C₁-C₂₀ alkyl, substituted or unsubstituted C₂-C₂₀ alkenyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}. In some examples of Formula I or Formula IA, R² is a 6-(2-(methylamino)-2-oxoethoxy)-N-phenyl-2-naphthamide group.

In some examples of Formula I or Formula IA, R² and R⁴ are the same.

In some examples of Formula I or Formula IA, R² and R⁴ are the same and are each substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}.

In some examples of Formula I or Formula IA, R² and R⁴ are the same and are each substituted or unsubstituted C₁-C₂₀ alkyl, substituted or unsubstituted C₂-C₂₀ alkenyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}.

In some examples of Formula I or Formula IA, R² and R⁴ are the same and are each substituted or unsubstituted C₃-C₂₀ aryl, substituted or unsubstituted C₄-C₂₀ alkylaryl, substituted or unsubstituted C₃-C₂₀ heteroaryl, or substituted or unsubstituted C₃-C₂₀ cycloalkyl.

In some examples of Formula I or Formula IA, R² and R⁴ are the same and are each substituted or unsubstituted C₃-C₂₀ aryl.

In some examples of Formula I or Formula IA, R² and R⁴ are the same and are each a substituted or unsubstituted C₆ aryl group (e.g., substituted or unsubstituted phenyl). In some examples, of Formula I or Formula IA, R² and R⁴ are the same and are each a substituted C₆ aryl group (e.g., substituted phenyl). In some examples of Formula I or Formula IA, R² and R⁴ are the same and are each an alkoxy substituted C₆ aryl group (e.g., alkoxy substituted phenyl). In some examples of Formula I or Formula IA, R² and R⁴ are the same and are each a 1,2,3-trimethoxybenzyl group.

In some examples of Formula I or Formula IA, the STAT5 inhibitor comprises StafiA-1, StafiB-1, or a derivative thereof.

In some examples, the STAT5 inhibitor comprises StafiA-1, StafiB-1, a derivative thereof, or a combination thereof.

In some embodiments, the STAT5 inhibitor is a StafiA-1. In some embodiments, the STAT5 inhibitor is a StafiB-1. In some embodiments, the STAT5 inhibitor is a pimozide.

In some embodiments, the STAT5 inhibitor is administered or applied.

The word “STAT5” includes both a STAT5A and a STAT5B. In some embodiments, the STAT5 is a STAT5A. In some embodiments, the STAT5 is STAT5B. “STAT5A” refers herein to a polypeptide that, in humans, is encoded by the STAT5A gene. In some embodiments, the STAT5A polypeptide, or its encoding polynucleotide, is that identified in one or more publicly available databases as follows: HGNC: 11366, Entrez Gene: 6776, Ensembl: ENSG00000126561, OMIM: 601511, UniProtKB: P42229. In some embodiments, the STAT5A polypeptide comprises the sequence of SEQ ID NO: 1, or a polypeptide sequence having at or greater than about 80%, about 85%, about 90%, about 95%, or about 98% identity with SEQ ID NO: 1, or a polypeptide comprising a portion of SEQ ID NO: 1. The STAT5A polypeptide of SEQ ID NO:1 may represent an immature or pre-processed form of mature STAT5A, and accordingly, included herein are mature or processed portions of the STAT5A polypeptide in SEQ ID NO: 1. “STAT5B” refers herein to a polypeptide that, in humans, is encoded by the STAT5Bgene. In some embodiments, the STAT5B polypeptide, or its encoding polynucleotide, is that identified in one or more publicly available databases as follows: HGNC: 11367, Entrez Gene: 6777, Ensembl: ENSG00000173757, OMIM: 604260, UniProtKB: P51692. In some embodiments, the STAT5B polypeptide comprises the sequence of SEQ ID NO: 2, or a polypeptide sequence having at or greater than about 80%, about 85%, about 90%, about 95%, or about 98% identity with SEQ ID NO: 2, or a polypeptide comprising a portion of SEQ ID NO: 2. The STAT5B polypeptide of SEQ ID NO:2 may represent an immature or pre-processed form of mature STAT5B, and accordingly, included herein are mature or processed portions of the STAT5B polypeptide in SEQ ID NO: 2.

A “TERT inhibitor” refers herein to a composition that reduces TERT interaction or binding with STAT5, reduces TERT activation of STAT5, and/or reduces TERT binding and/or activation of a RUNX2 gene or promoter. The word “TERT” refers to a polypeptide that, in humans, is encoded by the TERT gene. In some embodiments, the TERT polypeptide, or its encoding polynucleotide, is that identified in one or more publicly available databases as follows: HGNC: 11730, Entrez Gene: 7015, Ensembl: ENSG00000164362, OMIM: 187270, UniProtKB: 014746. In some embodiments, the TERT polypeptide comprises the sequence of SEQ ID NO: 3, or a polypeptide sequence having at or greater than about 80%, about 85%, about 90%, about 95%, or about 98% identity with SEQ ID NO: 3, or a polypeptide comprising a portion of SEQ ID NO: 3. The TERT polypeptide of SEQ ID NO: 3 may represent an immature or pre-processed form of mature TERT and accordingly, included herein are mature or processed portions of the TERT polypeptide in SEQ ID NO: 3. Several non-limiting examples of TERT inhibitors are imetelstat (GRN163L), BIBR1532, sodium metaarsenite (KML001), telomestatin, 6-thio-2′-deoxyguanosine (6-thio-dG), and XAV939.

In some embodiments, the STAT5 inhibitor and/or the TERT inhibitor reduces osteogenic differentiation of a cell. In some embodiments, the STAT5 inhibitor and/or the TERT inhibitor reduces osteogenic differentiation of vascular cells (valve cells, arterial cells). Included herein is data showing that TERT contributes to the osteogenic transition of primary aortic valve interstitial cells (AVICs). We utilized primary human aortic valve tissues from healthy control donors and calcific aortic valve disease (CAVD) patients and established patient-specific AVIC lines for in vitro disease modeling. We established baseline patterns and osteogenic induction of TERT and calcification markers in CAVD tissue and AVICs, assessed the consequences of genetic depletion of TERT in several cell systems, and defined the underlying mechanism by which TERT participates in the osteogenic transition of AVICs. We found that in diseased tissue, TERT protein was highly expressed in calcified areas without changes in telomere length, DNA damage nor senescence. AVICs isolated from diseased tissue retained a calcific phenotype. Genetic reduction of TERT prevented calcification and TERT levels were increased with osteogenic or inflammatory stimuli. We found that TERT and Signal Transducer and Activator of Transcription 5A/B (STAT5) colocalize and bind to the Runt-Related Transcription Factor 2 (RUNX2) gene promote and that STAT5 localize to cells expressing TERT in human tissue. Specific pharmacological inhibition of STAT5A isoform prevented AVICs calcification. These data show that TERT is required for calcification, TERT-directed transcriptional activity does not require telomere-elongation activity, regulates the transition of quiescent AVICs into calcifying AVICs, and that STAT5A functions as a TERT-interacting partner for binding and activating the RUNX2 gene promoter during calcification of AVICs. Accordingly, the methods of the present invention can, in some embodiments, be used to treat calcific aortic heart disease. In other or further embodiments, the present invention can be used to treat calcification in smooth muscle cells.

As noted herein, a STAT5 inhibitor and/or a TERT inhibitor can be administered to a subject via any route. In one embodiment, the administration is intravenous. In some aspects, the vein of the subject is selected from the group consisting of a subclavian vein, a thoracic internal vein, an axillary vein, a cephalic vein, a brachial vein, a basilic vein, an intercostal vein, a median cubital vein, a cephalic vein, an ulnar vein, a median antebrachial vein, and a palmar vein.

In some embodiments, the STAT5 inhibitor and/or a TERT inhibitor is administered at a dose of about 0.01 mg/kg of body weight, about 0.05 mg/kg of body weight, about 0.1 mg/kg of body weight, about 0.15 mg/kg of body weight, about 0.2 mg/kg of body weight, about 0.25 mg/kg of body weight, about 0.3 mg/kg of body weight, about 0.35 mg/kg of body weight, about 0.4 mg/kg of body weight, about 0.45 mg/kg of body weight, about 0.5 mg/kg of body weight, about 0.55 mg/kg of body weight, about 0.6 mg/kg of body weight, about 0.65 mg/kg of body weight, about 0.7 mg/kg of body weight, about 0.75 mg/kg of body weight, about 0.8 mg/kg of body weight, about 0.85 mg/kg of body weight, about 0.9 mg/kg of body weight, about 0.95 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, or about 20 mg/kg of body weight. In some embodiments, the nanobody is administered at a dose of at least about 0.01 mg/kg of body weight (e.g., at least about 0.1 mg/kg of body weight, at least about 0.2 mg/kg of body weight, at least about 0.3 mg/kg of body weight, at least about 0.5 mg/kg of body weight, at least about 1.0 mg/kg of body weight, at least about 2 mg/kg of body weight, at least about 5 mg/kg of body weight, at least about 10 mg/kg of body weight, at least about 50 mg/kg of body weight, or at least about 100 mg/kg of body weight).

The disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of calcification; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of calcification.

Dosing frequency for a STAT5 inhibitor and/or a TERT inhibitor of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, twice a day, three times a day, four times a day, or five times a day. Administration can also be continuous and adjusted to maintaining a level of the compound within any desired and specified range.

The STAT5 inhibitor and/or a TERT inhibitor and/or a composition comprising both inhibitors may be administered in such amounts, time, and route deemed necessary in order to achieve the desired result. The exact amount of the STAT5 inhibitor and/or a TERT inhibitor and/or a composition comprising both inhibitors will vary from subject to subject, depending on the species, age, and general condition of the subject, the severity of the calcification, the particular inhibitor or composition, its mode of administration, its mode of activity, and the like. The STAT5 inhibitor and/or a TERT inhibitor and/or a composition comprising both inhibitors are preferably formulated in dosage unit form for ease of administration and uniformity of dosage. It will be understood, however, that the total daily usage of the STAT5 inhibitor and/or a TERT inhibitor and/or a composition comprising both inhibitors will be decided by the attending physician within the scope of sound medical judgment. The specific therapeutically effective dose level for any particular subject will depend upon a variety of factors including the type of calcification being treated and the severity of the calcification; the activity of the STAT5 inhibitor and/or a TERT inhibitor and/or a composition comprising both inhibitors employed; the specific STAT5 inhibitor and/or a TERT inhibitor and/or a composition comprising both inhibitors employed; the age, body weight, general health, sex and diet of the patient; the time of administration, route of administration, and rate of excretion of the specific STAT5 inhibitor and/or a TERT inhibitor and/or a composition comprising both inhibitors employed; the duration of the treatment; drugs used in combination or coincidental with the specific STAT5 inhibitor and/or a TERT inhibitor and/or a composition comprising both inhibitors employed; and like factors well known in the medical arts.

The exact amount of a STAT5 inhibitor and/or a TERT inhibitor and/or a composition comprising both inhibitors required to achieve a therapeutically effective amount will vary from subject to subject, depending on species, age, and general condition of a subject, severity of the side effects or calcification, identity of the particular STAT5 inhibitor and/or TERT inhibitor and/or composition comprising both inhibitors, mode of administration, and the like. The amount to be administered to, for example, a child or an adolescent can be determined by a medical practitioner or person skilled in the art and can be lower or the same as that administered to an adult.

Also disclosed herein are pharmaceutically-acceptable salts and prodrugs of the STAT5 inhibitors and/or a TERT inhibitors described herein. Pharmaceutically-acceptable salts include salts of the disclosed STAT5 inhibitors and/or a TERT inhibitors that are prepared with acids or bases, depending on the particular substituents found on the compounds. Under conditions where the STAT5 inhibitors and/or a TERT inhibitors disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Methods of Screening Small Molecule STAT5 and/or TERT Inhibitors

In one aspect, disclosed herein is a method of screening one or more small molecule inhibitors to inhibit or reduce calcification, the method comprising introducing a STAT5 polypeptide and a TERT polypeptide in the presence of the one or more small molecule inhibitors, identifying the one or more small molecule inhibitors that reduce or prevent interaction between the STAT5 and TERT polypeptides, and wherein the one or more small molecule inhibitors reduce calcification of a circulatory system member or calcification of a device within a subject's circulatory system.

In some embodiments, the method includes detecting and visualizing an interaction between the STAT5 and TERT polypeptides, wherein the small molecule inhibitor prevents or reduces the interaction between the STAT5 and TERT polypeptides.

In some embodiments, the small molecule inhibitor is a peptide, polypeptide, nucleic acid, composition, or compound.

In some embodiments, the method detects a visual signal when STAT5 and TERT polypeptides interact. In some embodiments, the visual signal is decreased in the presence of the small molecule inhibitor.

In some embodiments, the visual signal is decreased by 50% or more in the presence of the small molecule inhibitor. In some embodiments, the visual signal is decreased by 50%, 60%, 70%, 80%, 90%, 95%, 99%, or 100% in the presence of the small molecule inhibitor.

In some embodiments, the small molecule inhibitor treats or reduces calcification of a circulatory member or calcification of a device within a subject's circulatory system.

In some embodiments, the circulatory system member is a heart valve. In some embodiments, the circulatory system member is a heart tissue. In some embodiments, the circulatory system member is a heart vein or a heart artery. In some embodiments, the circulatory system member is a vein or an artery in any area of the body. In some embodiments, the device is a bioprosthetic heart valve. In some embodiments, the device is a stent. In some embodiments the device is a vascular graft. In some embodiments, the subject is an animal or a human.

### Devices

Also included herein are devices comprising a pharmaceutically effective amount of a STAT5 inhibitor and/or a TERT inhibitor. In some embodiments, the device is made according to a method described herein. In some embodiments, the device is a prosthetic heart valve. In some embodiments, the heart valve is a bioprosthetic heart valve. In some embodiments, the device is a stent. In some embodiments the device is a vascular graft. In some embodiments, the application of a STAT5 inhibitor and/or a TERT inhibitor is made prior to the device's placement in the circulatory system member of a subject.

The STAT5 inhibitor and/or a TERT inhibitor comprised within or applied to the device can be any as described herein. In some embodiments, the STAT5 inhibitor comprises a compound of Formula I, or a pharmaceutically acceptable salt or derivative thereof. In some embodiments, the STAT5 inhibitor comprises a compound of Formula IA, or a pharmaceutically acceptable salt or derivative thereof. In some embodiments, the STAT5 inhibitor comprises a StafiA-1, a StafiB-1, or a derivative thereof. In some embodiments the STAT5 inhibitor comprises a StafiA-1, or a derivative thereof. In some embodiments, the STAT5 inhibitor comprises a StafiB-1, or a derivative thereof. In some embodiments, the STAT5 inhibitor is selected from the group consisting of a StafiA-1, a StafiB-1, a pimozide, a nicotinoyl hydrazine, a BP-1-108, an AC-4-130, and a SF-1-088. In some embodiments, the STAT5 inhibitor comprises a pimozide, or a derivative thereof. In some embodiments, the STAT5 is a STAT5A. In some embodiments, the STAT5 is a STAT5B.

### Examples

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Telomerase Reverse Transcriptase is Required in Calcific Aortic Valve Disease

Telomerase reverse transcriptase (TERT) plays a critical role in elongating telomeres at the end of chromosomes. Non-canonical transcriptional regulatory functions of TERT have been identified in a variety of tissues and cells. TERT can interact with transcription factors to induce gene expression. TERT also couples with chromatin remodeling complexes to promote trans-differentiation in several cell types. Related to calcification, ectopic TERT expression primes human mesenchymal stem cells (hMSCs) to differentiate down the osteoblastic lineage, showing a role for TERT in the activation of osteogenic transcriptional programs. It has also been discovered a role for TERT in cardiovascular calcification pathogenesis. In calcified aortic valve tissue, TERT protein was highly expressed in calcified areas, with no changes in telomere length, DNA damage markers, or senescence. In human aortic valve interstitial cells (hVICs) in vitro, TERT levels were increased with osteogenic or inflammatory stimuli. Runt-related transcription factor 2 (RUNX2) is required for the calcification of smooth muscle cells (mSMCs) in mice, and hVICs cultured in osteogenic media display elevated levels of RUNX2.

Tissue Collection and Isolation. Surgical specimens from human were collected from subjects who were consented and enrolled in studies approved by the institutional review board (IRB) of the University of Pittsburgh per the Declaration of Helsinki. Personnel involved with specimen handling underwent extensive institutional training. Cadaveric tissues obtained via the Center for Organ Recovery and Education (CORE) were approved by the University of Pittsburgh Committee for Oversight of Research and Clinical Training Involving Decedents (CORID). Briefly, valves were collected from valve replacement surgeries or recovered from cadaveric organs and stored in cold Belzer UW Cold Storage Transplant Solution (Bridge to Life) at 4° C. for transporting. Aortic roots were excised and washed with sterile rinsing solution (sterile PBS supplemented with 2.5 μg/mL of fungicide, 0.05 mg/mL of gentamicin, and 5 μg/mL of bactericide). Leaflets were unbiasedly selected for AVIC isolation, Von Kossa staining for calcification, and snap freezing for RNA collection. Tissues were processed as close as possible to the time of extraction to guarantee the best yield of cell recovery,

Human cell isolation. Patient-specific lines were established by using the same valves for histopathology, RNA, and cell isolation. Primary aortic valve interstitial cells (AVICs) were obtained from the patient aortic valve. Briefly, leaflets were washed with PBS containing 10 mg/ml gentamicin (GIBCO) and 250 μg/ml fungizone (GIBCO) and dissociated with 0.1% collagenase II at 37 C and 5% of CO2 for 18 hours. Then, the tissue was further dissociated by gently mixing it by pipetting with a serological pipette to ensure the release of A VICs and then passed through a 0.70 μm filter to remove debris. Cells were pelleted and then resuspended in Dulbecco's Modified Eagle's Medium (DMEM) supplemented with 10% fetal bovine serum (FBS) and 1×penicillin-streptomycin (P/S). Human coronary artery smooth muscle cells (CASMC) were obtained from patient coronary. Briefly, vessels were washed with PBS containing 10 mg/ml gentamicin (GIBCO) and 250 μg/ml fungizone (GIBCO). Vessels were cut open to expose the lumen and intima and adventitia were gently scrapped. Vessels were then sectioned and dissociated with 0.1% collagenase II for 3 hrs. Cells were pelleted and then seed and expanded.

Cell Culture. AVICs lines were expanded in Dulbecco's Modified Eagle's Medium supplemented with 10% fetal bovine serum and 1×penicillin-streptomycin. Cells were used between passages 4 and 15. Growth media was changed every three days, and cells were split 1:2 when confluent. CASMC lines were expanded in smooth muscle media SMGM (CC-3181) supplemented with BULLETKIT (CC-4149). Human MSCs (PT-2501, Lonza) were expanded on Minimum Essential Medium alpha without nucleosides and supplemented with 10% fetal bovine serum and 1×penicillin-streptomycin and used between passages 4 and 10. Cells are routinely tested for mycoplasma contamination.

Osteogenic Assay. For osteogenic experiments, 250,000 cells per 9.5 cm² were seeded and treated with osteogenic media (Gibco Minimum Essential Medium alpha with nucleosides, 10% FBS, 1×P/S, 10 mM glycerol phosphate, 50 μM ascorbic acid 2-phosphate, and 100 nM dexamethasone). No treatment media consisted of Gibco Minimum Essential Medium alpha with nucleosides, 10% FBS, 1×P/S. Media was replaced every four days and prepared fresh every time. BiBR1532 (2981, Tocris) was added to the media at 1, 10, or 100 nM every two days during the OST treatment⁴. Inhibitors StafiA-1 and StafiB-1 (Sigma-Aldrich) were used at 10 μM and added every two days for the duration of the OST treatment. For inflammatory assays, we used 2.5 μg/mL of LPS from Escherichia coli O111:B4 (Sigma-Aldrich) every two days for the duration of the OST treatment.

SDS-Page and immunoblotting. Cells were lysed in lysis buffer (1% CHAPS hydrate, 150 mM sodium chloride, 25 mM HEPES buffer), supplemented with 1× protease and phosphatase inhibitor cocktail (Sigma-Aldrich). Cells were scraped and transferred into microcentrifuge tubes, vortexed for 10 minutes, freeze/thawed for 5 cycles, then centrifuged at 12,000×g for 10 minutes at 4° C., and supernatants were collected. Proteins were separated with TGX 4-20% stain-free polyacrylamide gel (Bio-Rad) in 1× Tris/Glycine/SDS buffer (Bio-Rad) and transferred to 0.2 um nitrocellulose (1620112, Bio-Rad) membrane in 1× Trans-Blot Turbo Transfer Buffer (Bio-Rad) using the Trans-Blot Turbo Transfer System (Bio-Rad) according to the manufacturer recommendations. The membranes were blocked in Odyssey blocking buffer (PBS) (Li-COR) and immunoblotted overnight with primary antibodies against TERT (600-401-252, Rockland), STAT5 (9420S, Cell Signaling), RUNX2 (ab192256, Abcam), MYH11 (MAB20221, Abnova), OPN (AF808, R&D systems), αSMA (ab5494, Abcam), TNAP(MAB2909, R&D systems), a-tubulin (926-42213, LI-COR), followed by secondary anti-rabbit or anti-mouse IgG antibody (926-68070, 926-68021, 926-32211, LI-COR). Primary and secondary antibodies were diluted in Odyssey blocking buffer with 0.1% Tween 20. Membranes were washed in PBS with 0.1% Tween 20. Immunofluorescence signals were detected with the Odyssey CLx system (LI-COR), and images were analyzed with Image Studio (Version 5.2, LI-COR).

Immunofluorescent Staining on Fixed Cells. Cells were washed with PBS and then fixed with 4% paraformaldehyde (Electron Microscopy Sciences) for 15 minutes at room temperature, followed by 10 minutes of permeabilization with TRITON X-100 0.5% (Fisher Scientific). Cells were incubated at room temperature for 1 hour in blocking buffer (PBS, 0.5% Triton-X 100, 5% FBS). Primary antibodies were diluted in the blocking solution and then incubated overnight at 4° C. Next, cells were washed three times for five minutes each with PBS containing 0.1% TWEEN. Fluorescent secondary antibodies were diluted in blocking solution and incubated for one hour protected from the light, then washed three times for five minutes each with PBS, 0.1% TWEEN 20 with a final wash in PBS. Specimens were finally mounted with Fluoroshield Mounting Medium with DAPI (Abcam) and imaged within 24 hours. F-Actin was stained with AlexaFluor488 Phalloidin (Molecular Probes) for 30 minutes, then washed with PBS before mounting. Calcium accumulation was determined with OsteoImage (Lonza) following the manufacturer's recommendations.

Immunofluorescent Staining on Paraffin Sections. Human specimens embedded in paraffin were warmed to 65° C. and deparaffinized using xylene and graded alcohol baths, rehydrated in distilled H2O, and boiled for 20 minutes in antigen unmasking solution (Vector Labs). Cooled samples were then washed with PBS for five minutes. Specimens were then blocked with PBS containing 3% fish skin gelatin and 10% horse serum for one hour. Primary antibodies were diluted in the blocking solution and then incubated overnight at 4° C. Next, specimens were washed three times for five minutes each with PBS containing 3% fish skin gelatin and 0.1% TWEEN 20. Fluorescent secondary antibodies were diluted in blocking solution and incubated for one hour protected from the light, then washed three times for five minutes each. Specimens were finally mounted with Fluoroshield Mounting Medium with DAPI (Abcam) and imaged within 24 hours.

Alizarin Red Staining and Quantification. Cells were washed with PBS and then fixed with 4% paraformaldehyde (Electron Microscopy Sciences) for 15 minutes at room temperature, followed by two washes with deionized water. Fixed cells were then covered with 40 mM Alizarin Red S (Sigma-Aldrich) at pH 4.1-4.3 and gently rocked for 20 minutes at room temperature. Cells were then washed twice with deionized water to remove any unincorporated dye. After imaging, Alizarin Red S was extracted with 10% (v/v) acetic acid (Fisher Scientific) for 30 minutes, scraped into a microcentrifuge tube, vortexed, and then incubated at 85° C. for 10 minutes. After chilling on ice for 5 minutes, the mixture was centrifuged at 20,000×g for 15 minutes at 4° C. 500 μL of supernatant was transferred to a new tube, and 10% (v/v) ammonium hydroxide (Fisher Scientific) was then added to the supernatant. Absorbance was at 405 nm using a 96-well plate spectrophotometer.

Von Kossa Staining. Human specimens embedded in paraffin were warmed to 65° C. for one hour and then deparaffinized through xylene and graded alcohol incubations. Specimens were then rehydrated in distilled water. Specimens were stained using the Von Kossa Method of Calcium Kit (Polysciences, 24633-1) following the manufacturer's instructions.

Lentiviral Production and Cell Infection. Lentiviruses were produced by transfecting Dharmacon SMARTvector Lentiviral plasmids encoding shTERT TurboGFP (V3SH11240-225610522) or SMARTvector Non-targeting Control (VSC11707) into HEK293T cells using FuGENE 6 (Promega). The viral-containing supernatant was collected at 48 h after transfection, filtered through a 0.45-μm filter, and stored at −80C. Human SMC and AVICs lines were transduced with MOI of 5 in the presence of 0.8 μg ml⁻¹ polybrene (Millipore) to enhance transduction efficiency.

Transcriptional Analysis. Tissue RNA was isolated using Trizol (Life Technologies). Cell RNA was isolated using Quick-RNA MiniPrep (Zymo Research). RNA was treated with DNAse I (Zymo Research) in accordance with the manufacturer's instructions. Reverse transcription was performed using MultiScribe Reverse Transcriptase system (Applied Biosystems). Sixteen ng of cDNA was used per reaction. qPCR was performed on a CFX Connect Real-Time System (Bio-Rad) using PowerUP SYBR Green Master Mix (Applied Biosystems) as follows: one cycle at 95° C. (10 minutes) and 40 cycles of 95° C. (20 seconds) and 58° C. (20 seconds) and 72° C. (1 minutes). GAPDH or 18s expression were used to normalize expression. Relative expression was calculated using the average threshold cycle number and the 2^{((Ct(housekeeping gene)-Ct(target gene))} formula. Primers are listed in Table 4.

Telomere Length Analysis. Tissue and AVICs genomic DNA was isolated from passage 1 using DirectAmp Tissue Genomic DNA Amplification Kit (Denville Scientific). Telomere length was analyzed using real-time PCR as previously described. Briefly, genomic DNA was isolated following standard protocol, and 10 ng of gDNA per reaction was utilized. Samples were run in triplicate with 35 ng of DNA per reaction, and telomere repeats were amplified using PowerUP SYBR Green Master Mix (Thermofisher Scientific) on a CFX Connect Real-Time PCR System (Bio-Rad). Repeated amplification data were normalized to RPLP0/36b4 as a single copy-gene. Primers are listed in Table 4.

Proliferation assays. Cell proliferation was evaluated using Trypan Blue incorporation in an automatized cell counter Countess II FL (Invitrogen). Briefly, cells were grown on alpha-MEM supplemented with 10% of FBS and penicillin and streptomycin cocktail (GIBCO) for the duration of the assay. Growth was quantified twice a week. Cell number during OST treatment was determined at the beginning and end of the assay.

Senescence-Associated β-Galactosidase Assays. Cellular senescence was evaluated by senescence-associated β-Galactosidase (SA-β-Gal) activity assay. Briefly, cells were washed with PBS and then fixed with 4% paraformaldehyde (Electron Microscopy Sciences) for 5 minutes at room temperature and then washed twice with PBS. Next, cells were incubated in X-Gal Solution Mix containing 1 mg/mL of 5-Bromo-4-Chloro-3-Indolyl-Alpha-D-Galactopyranoside (American Bioanalytical), 40 mM Sodium Phosphate monobasic solution (Sigma-Aldrich), 40 mM Sodium Phosphate Dibasic solution (Sigma-Aldrich), 40 mM Citric Acid (Sigma-Aldrich), 5 mM Potassium Ferrocyanaide (Sigma-Aldrich), 5 mM Potassium Ferricyanaide (Sigma-Aldrich), 150 mM NaCl₂, and 2 mM MgCl₂, pH 6.0, for 16 hours at 37° C. without CO₂ to develop blue color. Then, cells were washed twice with PBS, and SA-β-gal positive cells were imaged and calculated based on randomly selected bright fields areas containing at least 200 cells per field as revelated by subsequent DAPI staining.

Cell Migration Assay. AVICs were seeded in a 12-well plate at a concentration of 1×10⁵ cells per well and were left until they reached 90% of confluence. The well surface was scratched with a 200 μL sterile pipette tip and washed with PBS to removed detached cells. Horizontal lines were drawn on the bottom outside of the well and used as a reference for alignment to obtain the same field for each image acquisition run. AVICs media was added, and images were collected at different time points with a phase-contrast microscope using as guide the reference marks. Scratch area and scratch width were determined with the Wound Healing Size Macro Tool using ImageJ. Linear regression and two-way ANOVA to compare the changes in the area and the average length of the scratch.

Chromatin Immunoprecipitation. Chromatin Immunoprecipitation (ChIP) was performed on cultured cells. Briefly, AVICs were stimulated with osteogenic media for 14 days and then collected in 20 mM Na-butyrate dissolved in PBS, and cross-linking of DNA and proteins was performed with formaldehyde (1% vol/vol final concentration) at room temperature. Cross-linking was stopped with 125 mM glycine for 10 min. Cross-linked chromatin was sonicated to obtain fragments between 250 and 750 base pairs in a Bioruptor Pico sonication device (Diagenode). The sheared chromatin was immunoprecipitated with 1 ug of antibody raised against TERT (Rockland, 600-401-252S) and 0.3 ug STAT5 (Cell Signaling, 94205S). Normal rabbit polyclonal IgG (Cell Signaling, 2729S) was used as a negative control. Negative control was incubated with rabbit IgG and input DNA primary antibody. Chromatin complexes were recovered with ChIP-grade Protein G magnetic beads (9006S, Cell Signaling). DNA was recovered with standard phenol-chloroform extraction. Immunoprecipitated DNA was amplified by quantitative RT-PCR using SYBR green. ChIP primers are listed in Table 4.

Proximity Ligation Assay (PLA). PLA was performed directly after cell fixation and according to manufacturer instructions. Briefly, cells were blocked with Duolink blocking solution for 60 minutes at 37° C. Samples were then incubated with rabbit TERT (Rockland) and mouse STAT5 (Santa Cruz) antibodies diluted in Duolink Blocking Solution and incubated overnight at 4° C. Samples were then incubated with secondary antibodies conjugated with PLA probes (DUO92002, DUO92004, Sigma) for 2 h at 37° C. in a humidity chamber followed by probe ligation for 30 minutes at 37° C. as recommended by the manufacturer. Amplification was performed with Duolink detection kit Red 595 nm for 100 minutes at 37° C. (DUO92101, Sigma). Samples were mounted in DAPI-containing mounting media and prepared for image acquisition.

Murine cell isolation. Tert knockout mice (Jackson Labs, 005423) were bred with wild-type mice (Jackson Labs, 000664) to produce Tert heterozygous mice, which were then bred to each other to yield Tert knockout mice and wild-type littermate controls. Het-het breeding ensures telomeres are intact. Mouse BMMSCs were isolated from femurs and tibias were dissected from three-month-old knockout or wild-type mice. The marrow was rinsed out of the bones with MSC media, and cells were plated and expanded as described previously⁸. Mouse AVICs were isolated from hearts from three-month-old Tert knockout or wild-type mice were removed, dissected, and valve leaflet removed. Cells were isolated and expanded. Mice were given veterinary care by the University of Pittsburgh Division of Laboratory Animal Resources, which adheres to the NIH policy on the Animal Welfare Act and all other applicable laws. Facilities are under the full-time supervision of veterinarians and are AAALAC-accredited. These protocols follow the AVMA Guidelines on Euthanasia. All animal breeding and isolations were approved by the Institutional Animal Care and Use Committee at the University of Pittsburgh. Attempts were made to minimize the number of mice required to complete experiments.

Statistics. Statistical analyses were performed with GraphPad Prism 9.2 (GraphPad Software, San Diego, CA) software. All experiments used at least n=3 biological replicates and were run in technical duplicates. Statistical comparisons between the 2 groups were performed by nonparametric Mann-Whitney U test. Statistical comparisons among 2 or more groups were performed by nonparametric Kruskal-Wallis H tests with post hoc Dunn's multiple pairwise comparisons between groups. Shapiro-Wilk test was used to test data distribution. Data are shown as mean±SD.

### Results

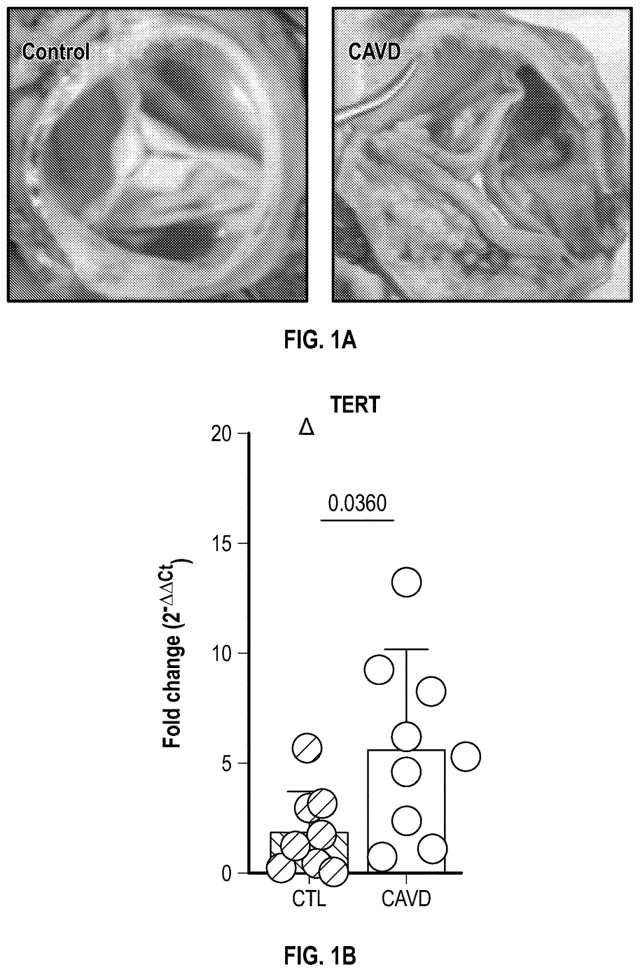

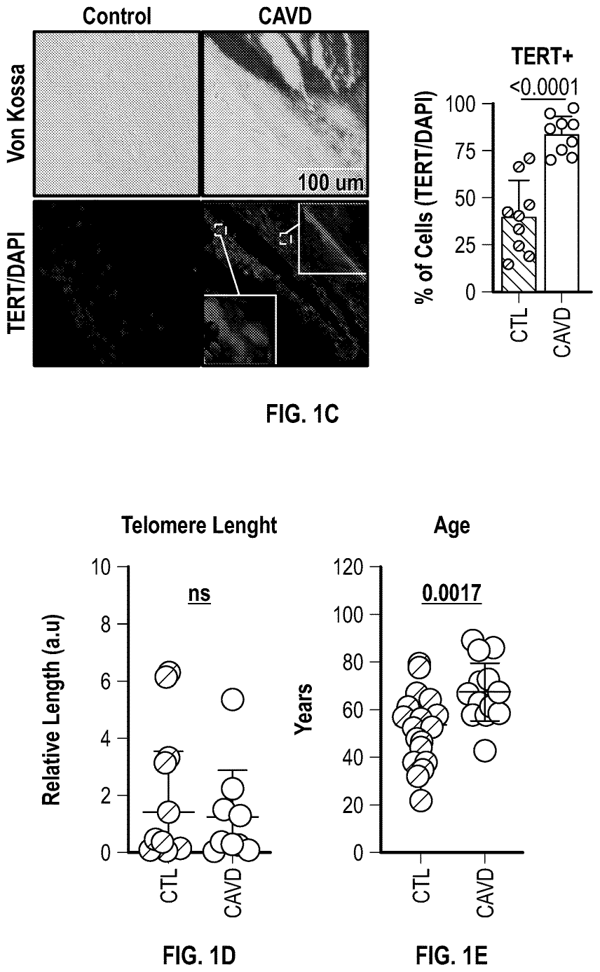

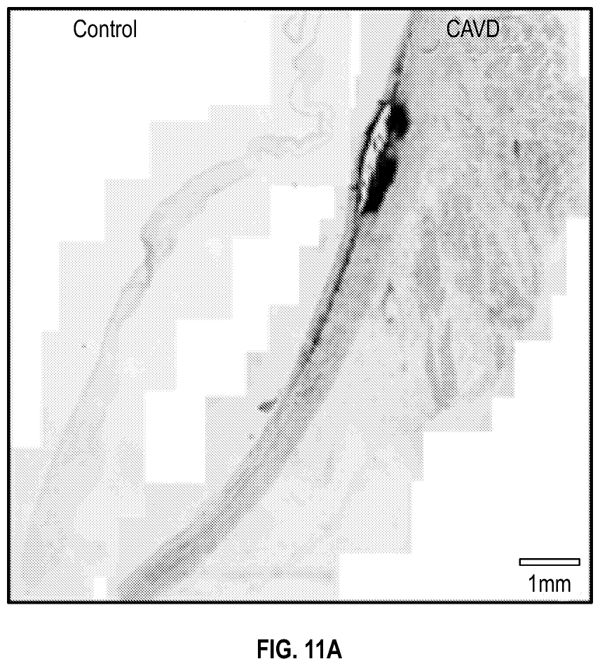

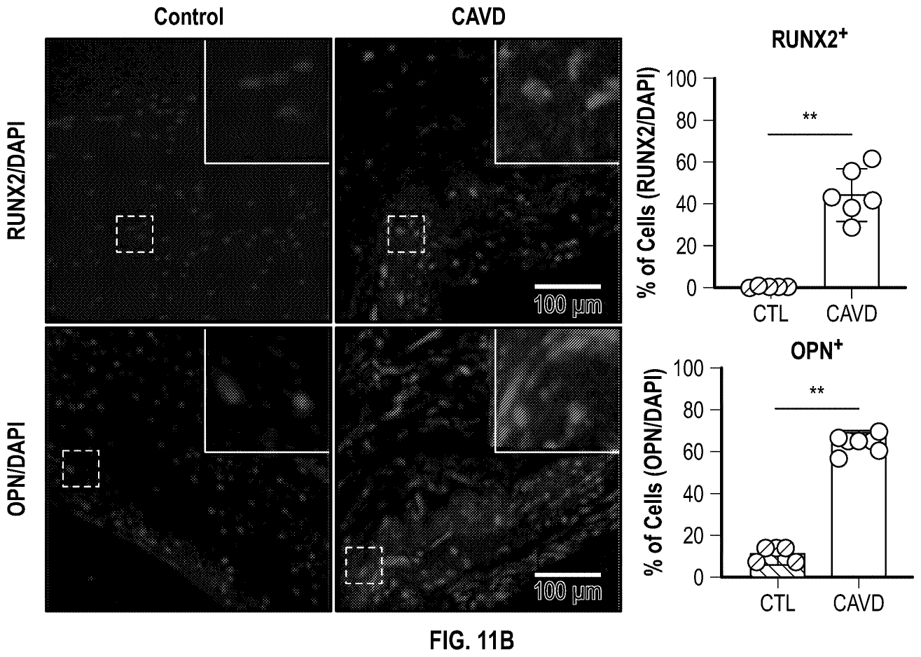

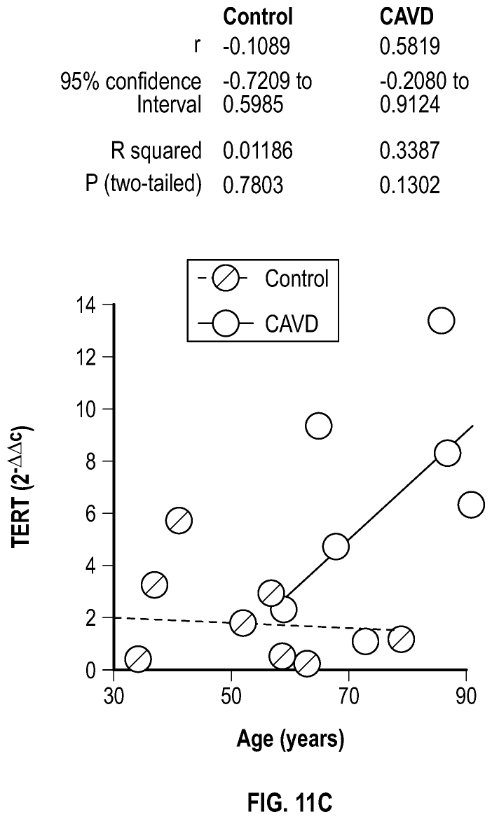

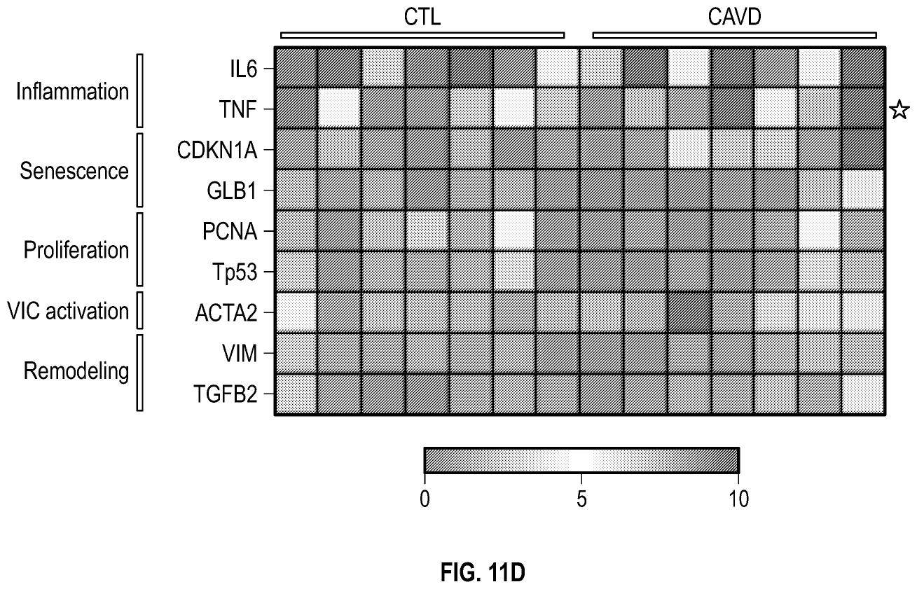

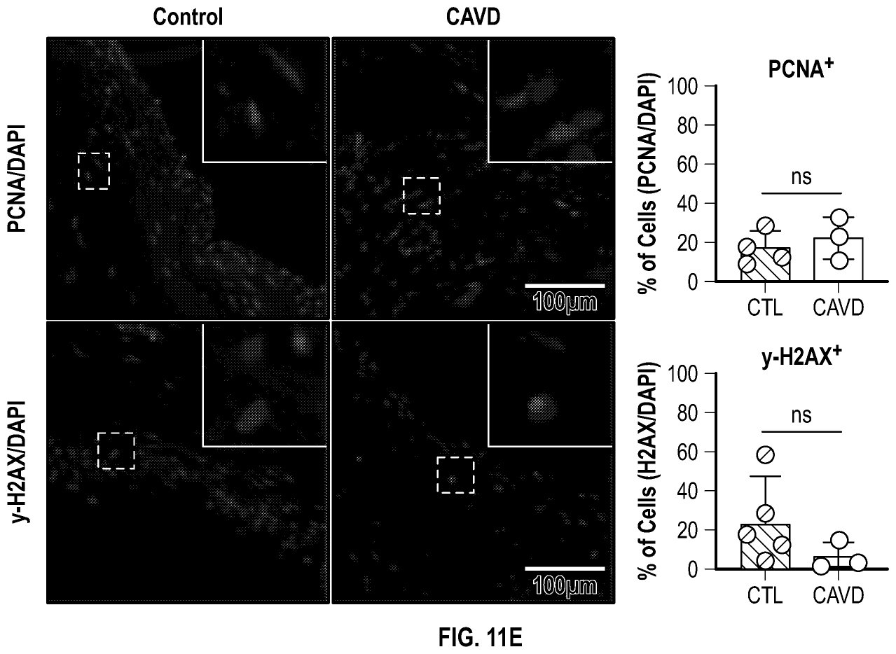

TERT is upregulated in CAVD valve tissue. Surgically removed CAVD leaflets exhibited extensive calcification and thickening compared to control non-calcified valves (FIG. 1A, FIG. 11A, and Table 1, patient information). Initial characterization of the specimens showed that the protein levels of the early calcification marker RUNX2 and the late calcification marker osteopontin (OPN) were elevated in CAVD tissue compared to controls (FIG. 11B). TERT transcript was significantly upregulated in CAVD tissue relative to control samples (FIG. 1B), and its levels correlated positively with the donor's age (FIG. 11C). The expression of several genes involved in pathways contributing to CAVD pathogenesis was examined including interleukin 6 and tumor necrosis factor alpha (IL6, TNF, respectively; indicative of inflammation); cyclin dependent kinase inhibitor 1A and galactosidase beta 1 (CDKN1A, GLB1, respectively; indicative of senescence); proliferating cell nuclear antigen and tumor protein 53 (PCNA, TP53, respectively; markers of proliferation and DNA damage respectively); actin alpha 2 smooth muscle/αSMA (ACTA2; indicative of AVIC activation); and vimentin and transforming growth factor beta 2 (VIM, TGFB2, respectively; indicative of matrix remodeling) (FIG. 11D). IL6 and TNF transcript expression was higher in CAVD valves. Combined, these data show that inflammatory signaling are involved in TERT activation, as shown by others.

Immunofluorescence staining revelated that TERT protein was localized to areas of calcification as determined by Von Kossa staining of serial sections, while the number of cells expressing TERT was significantly elevated in CAVD tissues compared to control tissues (FIG. 1C). No differences were detected between CAVD and control tissues in levels of the proliferating cell nuclear antigen (PCNA) marker or the double-stranded break indicator phosphorylated gamma histone 2AX (γ-H2AX) (FIG. 11E). The mean relative telomere length between CAVD and control tissues was indistinguishable (FIG. 1D), while the mean age of CAVD and control patients in this study was 68 and 54 years old, respectively (FIG. 1E). Together, these data show that TERT, osteogenic markers, and inflammatory signatures are upregulated in CAVD tissue while markers indicating alterations in proliferation/DNA damage/senescence are not altered in these CAVD tissues.

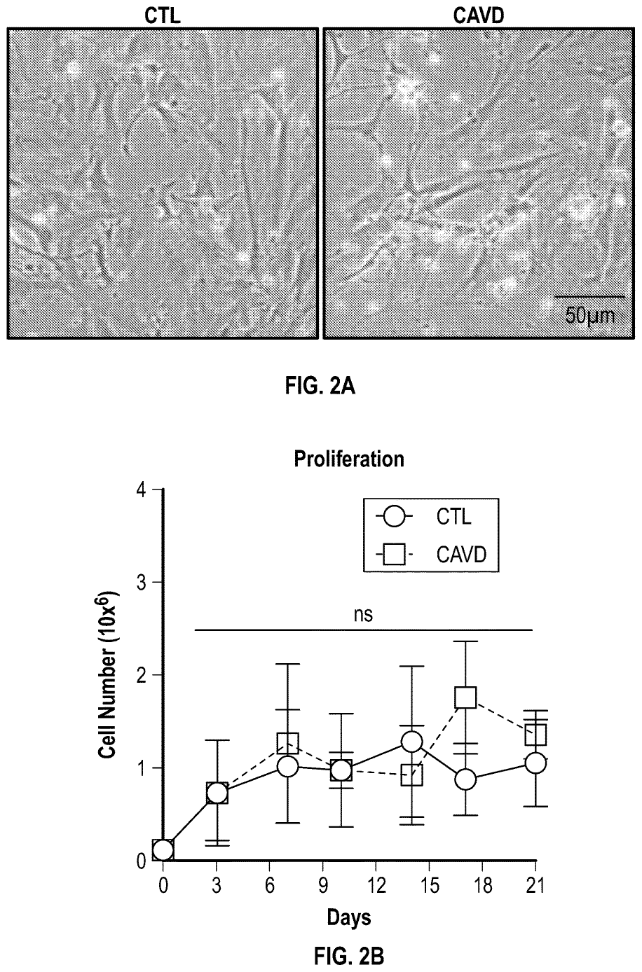

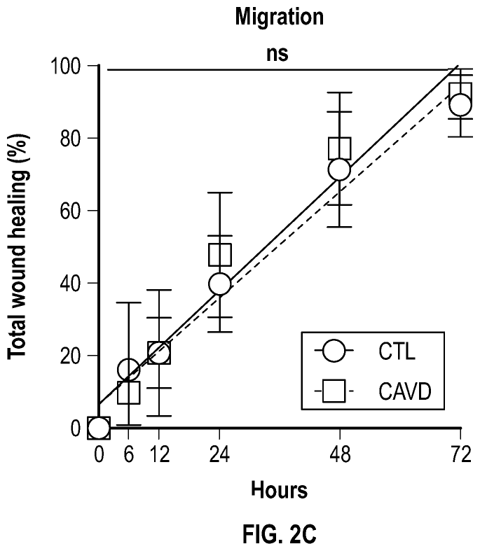

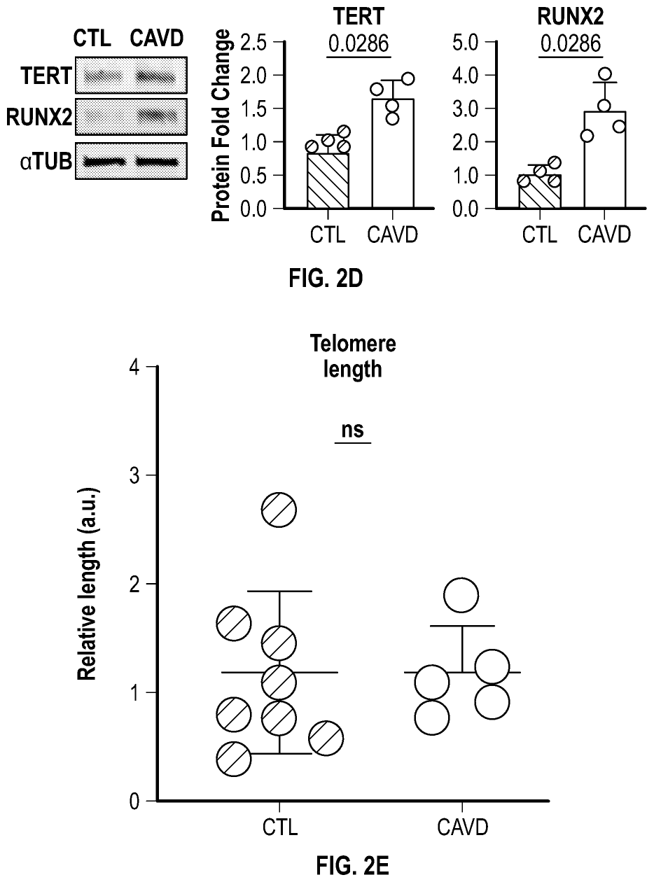

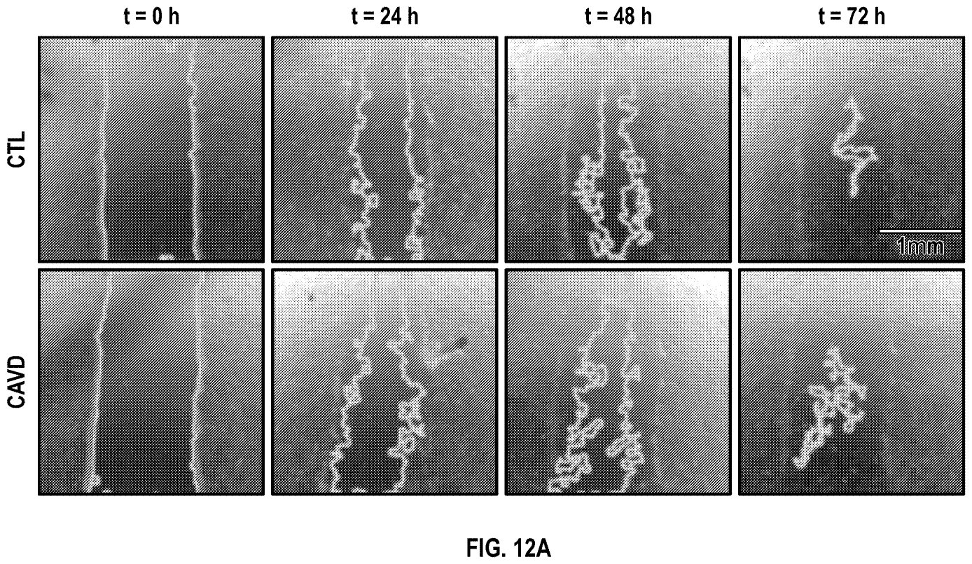

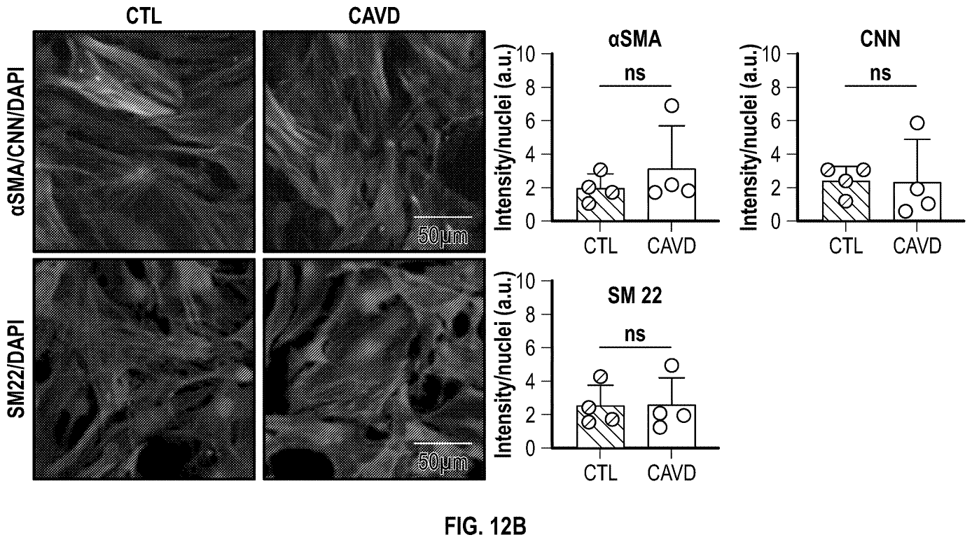

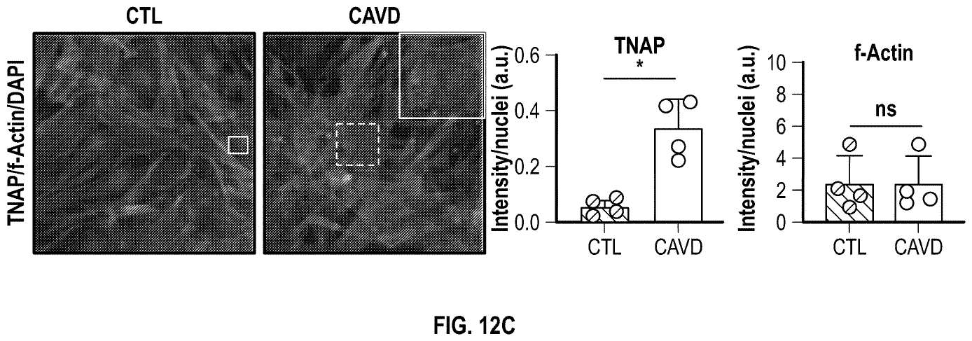

In vitro CAVD disease modeling with AVICs recapitulates in vivo observations. Patient-specific AVIC lines isolated from CAVD and control valves were established. At baseline, freshly isolated AVICs CAVD and control AVICs did not exhibit morphological (FIG. 2A), proliferative (FIG. 2B), or migratory differences (FIG. 2C, and FIG. 12A) while TERT and RUNX2 proteins were significantly elevated in CAVD AVICs (FIG. 2D). Also at baseline, no differences between CAVD and control AVICs were observed in SM22 or αSMA—markers of an activated AVIC phenotype (FIG. 12B)—but found significantly higher levels of the mineralizing enzyme tissue non-specific alkaline phosphatase (TNAP) in CAVD AVICs (FIG. 12C). Like in valve tissues, there was no difference in the mean telomere length between CAVD and control groups (FIG. 2E). Thus, these data show that at baseline, AVICs isolated from CAVD patients show elevated TERT and RUNX2 expression and recapitulate the osteogenic phenotype observed in CAVD valve tissue.

### TERT Expression is Increased Under Osteogenic Conditions In Vitro

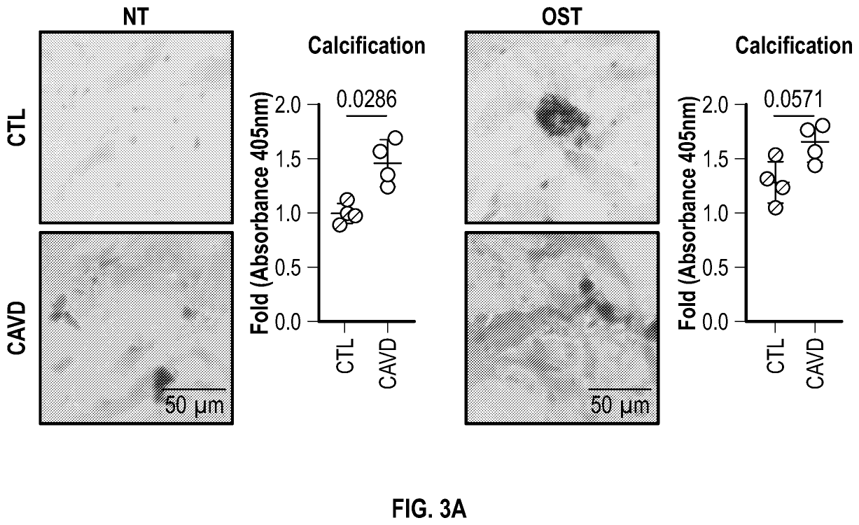

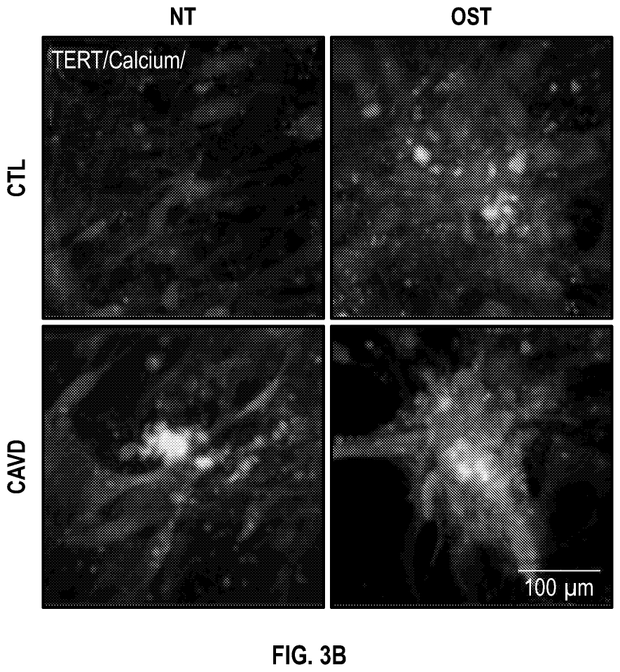

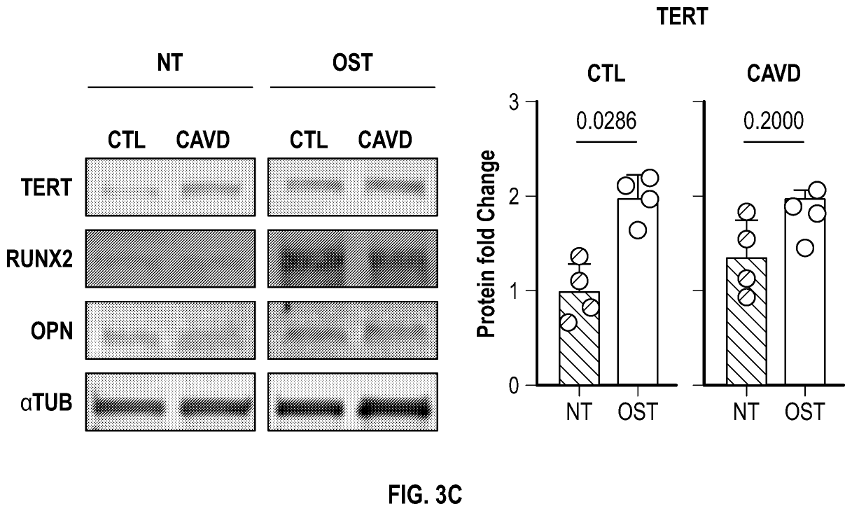

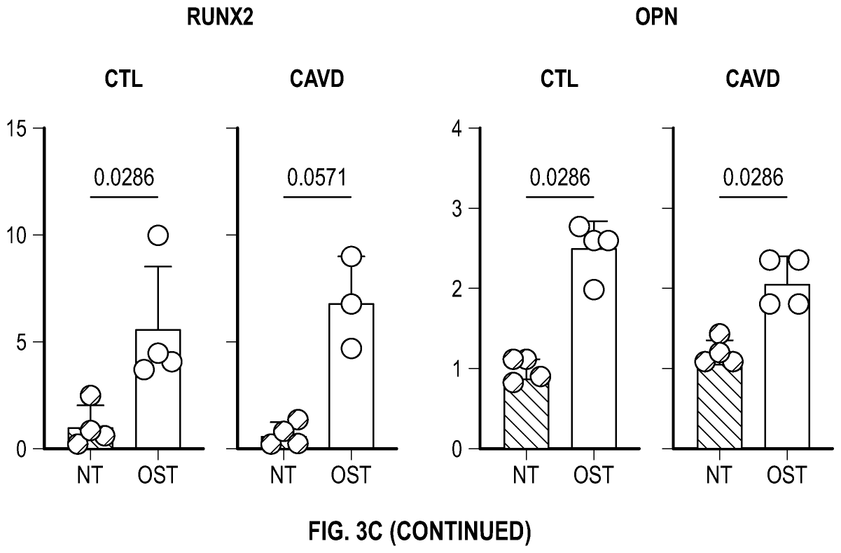

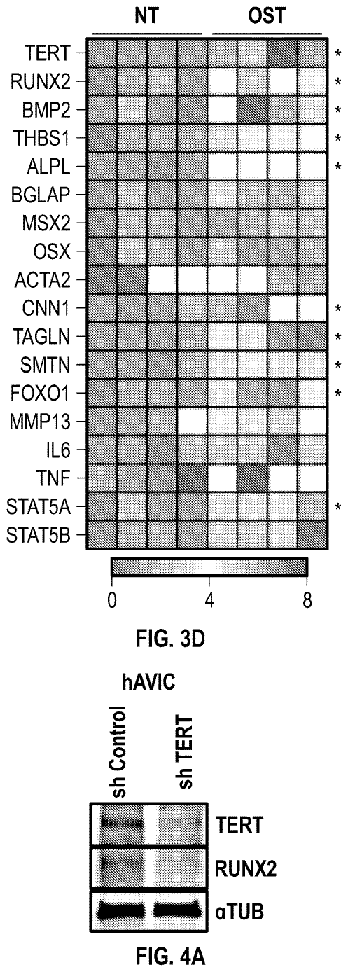

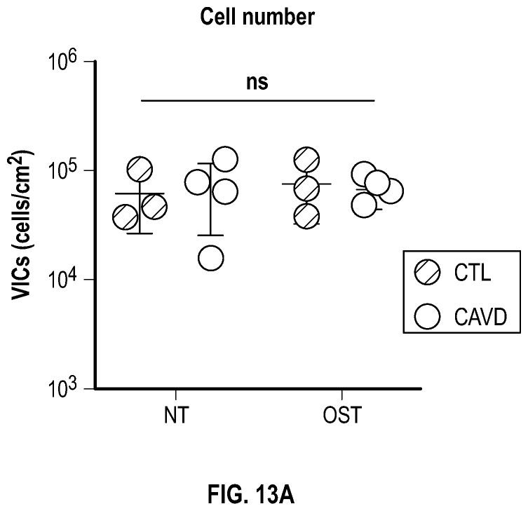

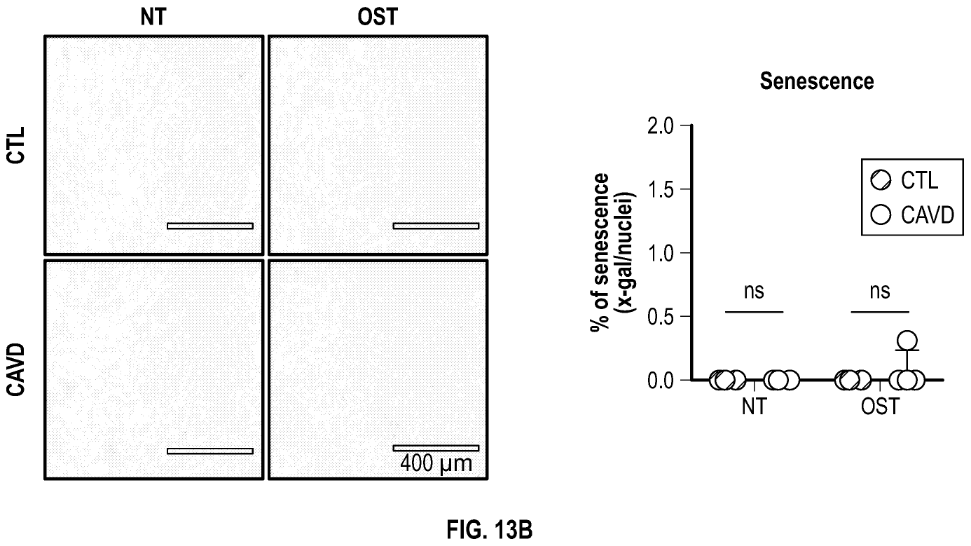

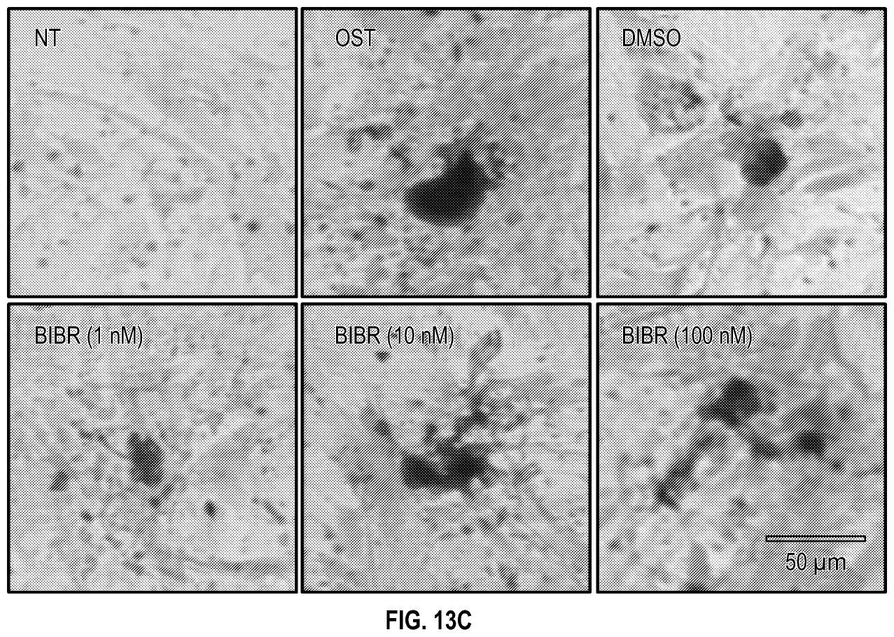

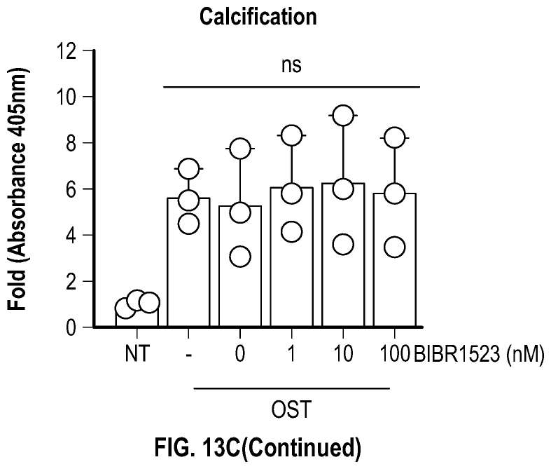

To model CAVD disease, CAVD and control AVICs were cultured in osteogenic media (OST). Alizarin Red staining revelated that OST treatment induced the calcification of CAVD and control A VICs as early as 14 days (d14) of treatment, and the calcification observed in CAVD AVICs was elevated relative to control AVICs under the same conditions, while it was also observed that CAVD AVICs calcify de novo (no treatment, NT, FIG. 3A). Immunofluorescent staining showed that at baseline, TERT was elevated in CAVD AVICs relative to control AVICs and that the signal intensified in both the cytosol and nucleus after 14 days of OST treatment. Furthermore, CAVD AVICs cluster was observed around calcified nodules while control A VICs exhibited moderate TERT staining, and no AVIC clustering was observed under the same conditions (FIG. 3B). No differences in cell numbers were observed at the end of the assay under any experimental conditions (FIG. 13A). Protein analysis revealed that the expression of TERT, RUNX2, and OPN was elevated in CAVD and control AVICs after 14 days of OST treatment (FIG. 3C). Transcriptional analysis showed that the osteogenic markers BMP2, THBS1, ALPL; myofibroblast markers CNN1, TAGLN, SMTN; remodeling-associated gene FOXO1; and the CAVD-associated metalloproteinase MMP-13 were significantly upregulated in control AVICs after osteogenic stimulation. Significant upregulation of the pro-inflammatory transcription factor Signal Transducer and Activator of Transcription 5A (STAT5A) was upregulated while the isoform STAT5B expression remained unchanged (FIG. 3D). We also observed that OST treatment did not induce senescence in AVICs, as determined by senescence-associated β-galactosidase (SA-β-gal) activity (FIG. 13B), recapitulating what was observed in the valve tissue (FIG. 1). Finally, to assess the involvement of the canonical, telomere extending, the activity of TERT in calcification, AVICs were treated with the TERT inhibitor BIBR1532. Several doses of the telomere-extending activity inhibitor BIBR1532 were added every two days during OST treatment, and it was found that even after 28 days of treatment, BIBR1532 did not alter the ability of AVICs to calcify (FIG. 13C). This last observation demonstrates that the canonical function of TERT is not operative in the calcification of AVICs. Altogether, this data show that TERT has a role during the osteogenic transition of AVICs.

### TERT is Required for In Vitro Calcification

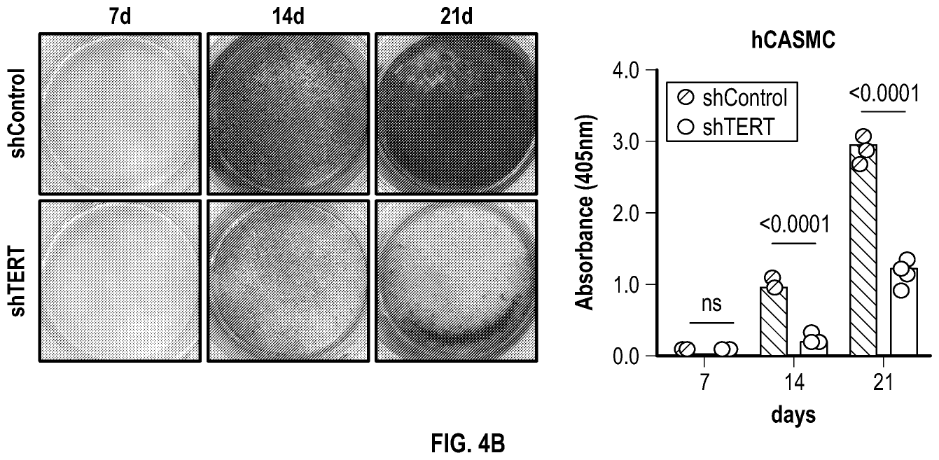

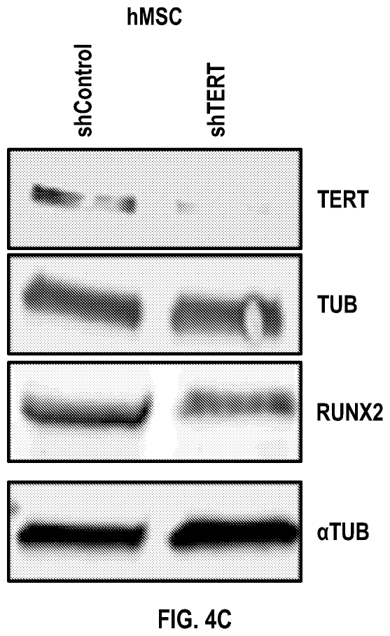

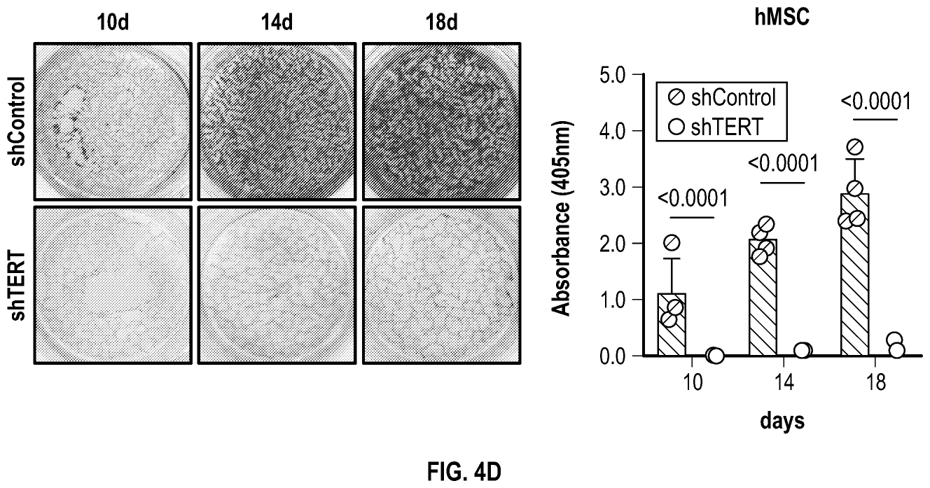

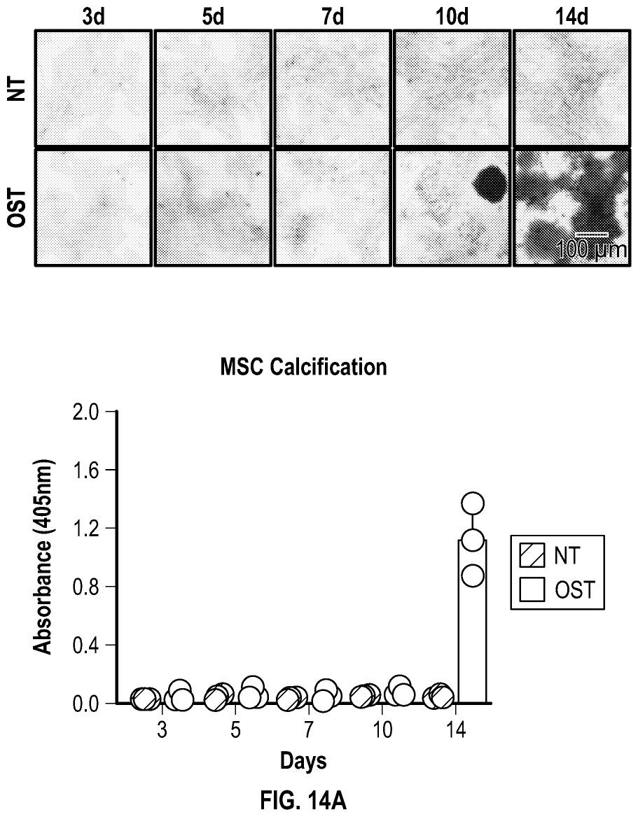

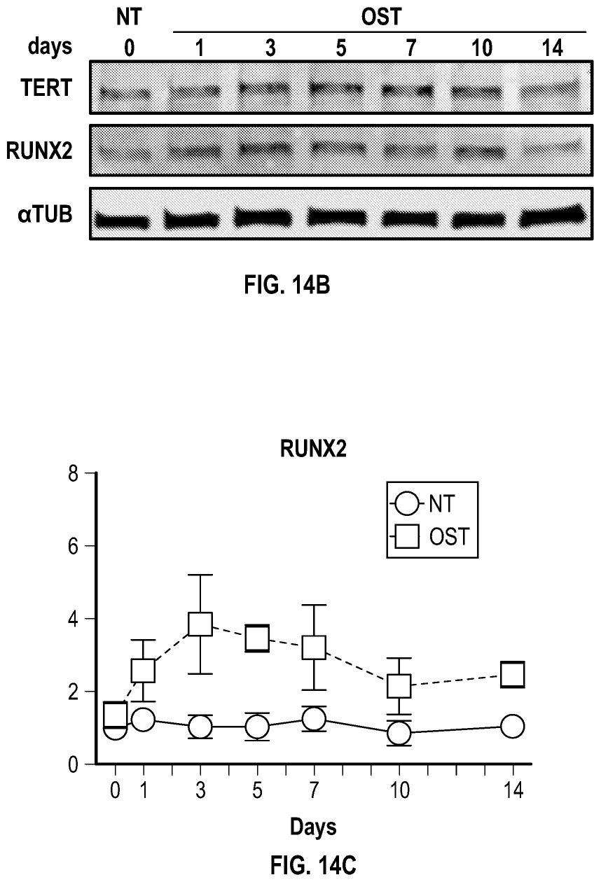

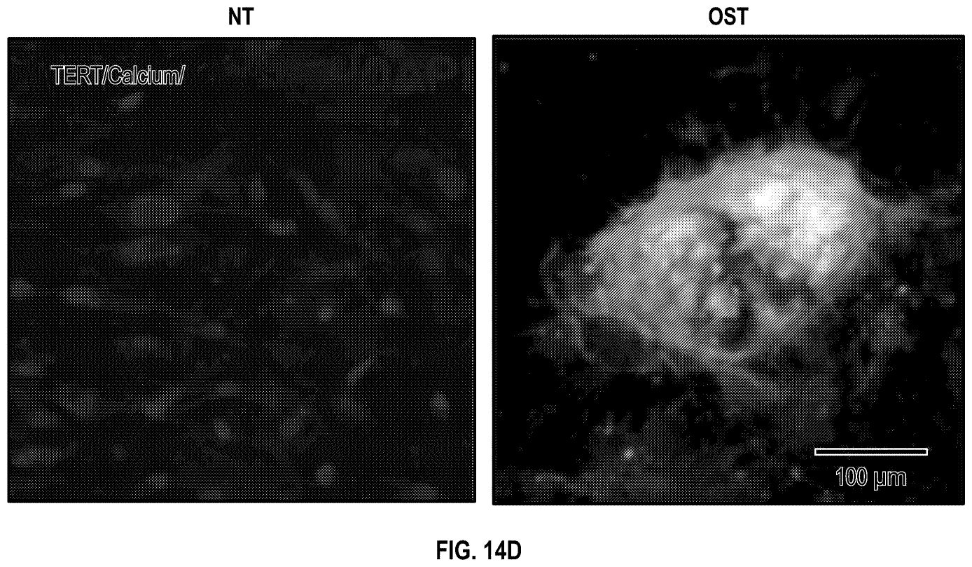

It has been shown that the overexpression of TERT primes human MSCs (hMSCs) to differentiate into osteoblasts. To determine whether TERT is required for the calcification of human AVICs, these cells were transduced with a lentivirus expressing an shRNA targeting TERT. Knockdown of TERT caused downregulation of RUNX2 in human AVICs (FIG. 4A). Next, it was assessed whether TERT depletion affects the calcification of human coronary artery smooth muscle cells (hCASMCs), as these vascular cells show a robust calcific phenotype. In hCASMCs, the knockdown of TERT inhibited calcification of hCASMC (FIG. 4B). Finally, to validate these findings, TERT expression was knocked down in human MSCs. Human MSCs exhibited robust calcification as early as d14 of the OST treatment, while cells in NT conditions did not calcify (FIG. 14A). Protein analysis showed TERT protein level increases on day 3 of the OST differentiation, coinciding with the increase in RUNX2 expression and RUNX2 protein levels (FIGS. 14B and 14C). Like AVICs, hMSCs in OST treatment exhibited an intense TERT staining, and TERT-positive cells clustered around calcified nodules (FIG. 4D). As previous assays, the knockdown of TERT caused downregulation of RUNX2 in MSCs while it also inhibited calcification (FIGS. 4C and 4D). Together, these data show that TERT is required for the osteogenic differentiation of human AVICs, CASMCs, and MSCs.

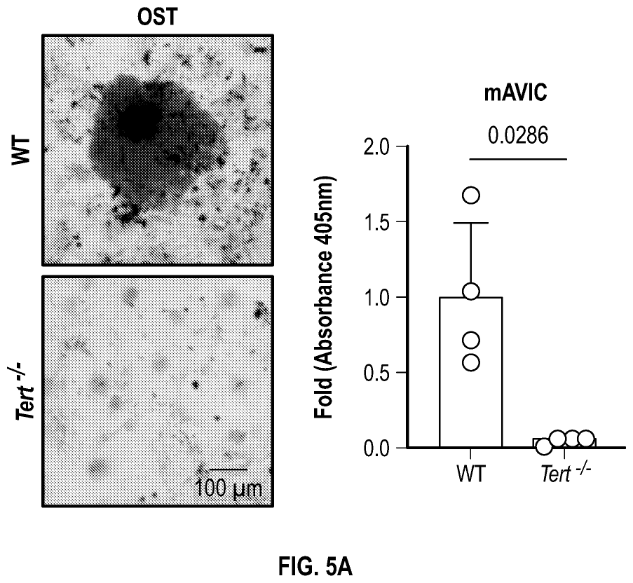

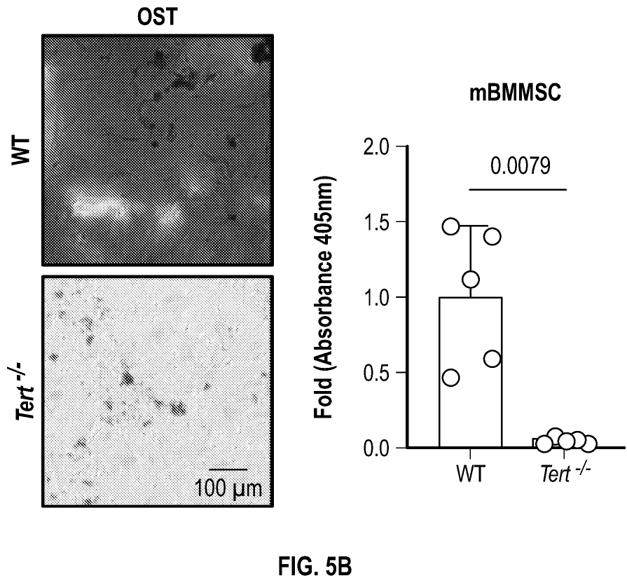

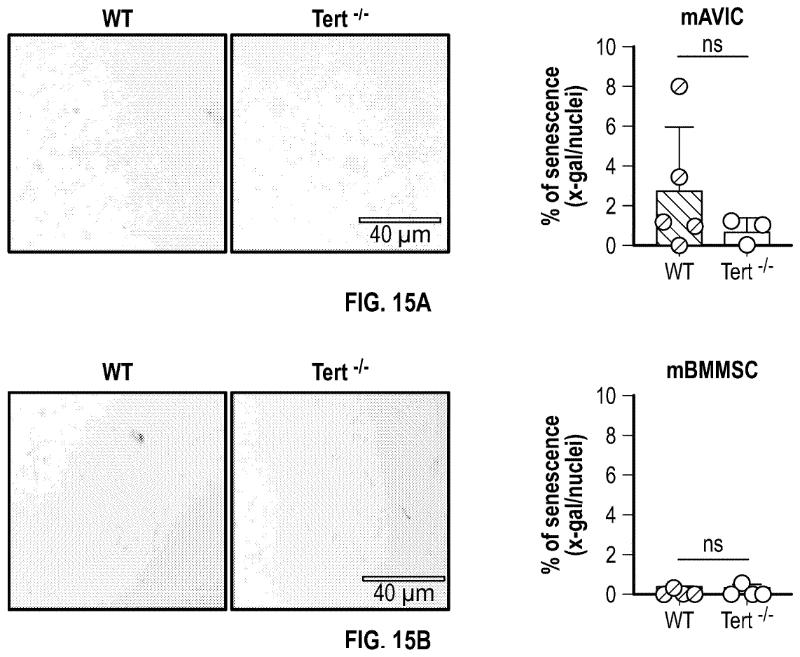

To further these findings that TERT is required for calcification, a genetic approach was used. Freshly isolated mice AVICs (mAVICs) and bone marrow MSCs (mBMMSCs) cells were isolated from Tert-knockout (Tert^{-/-}), and wild-type (WT) strain and calcification capacity was assessed. Importantly, Tert-knockout (Tert^{-/-}) mice were generated from heterozygous breeding pairs (F1 generation) and thus they do not exhibit shortened telomeres. In support of the findings in the human cells, deletion of TERT drastically inhibited calcification of mAVICs and mBMMSCs compared to WT cells, which exhibited robust calcification (FIGS. 5A and 5B). Notably, senescence in Tert^{-/-} cells was not detected as determined by SA-β-gal activity (FIGS. 15A and 15B). Together, these data show that TERT is required for the osteogenic transition of mice AVICs and BMMSCs, and that senescence is not operative during the osteogenic differentiation of murine cells.

TERT Interacts with STAT5 to Bind the RUNX2 Gene Promoter.

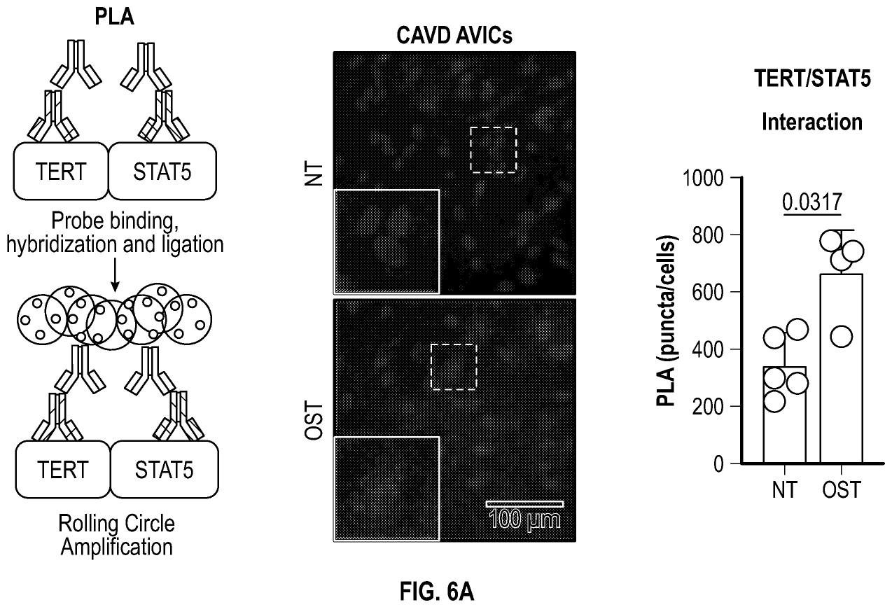

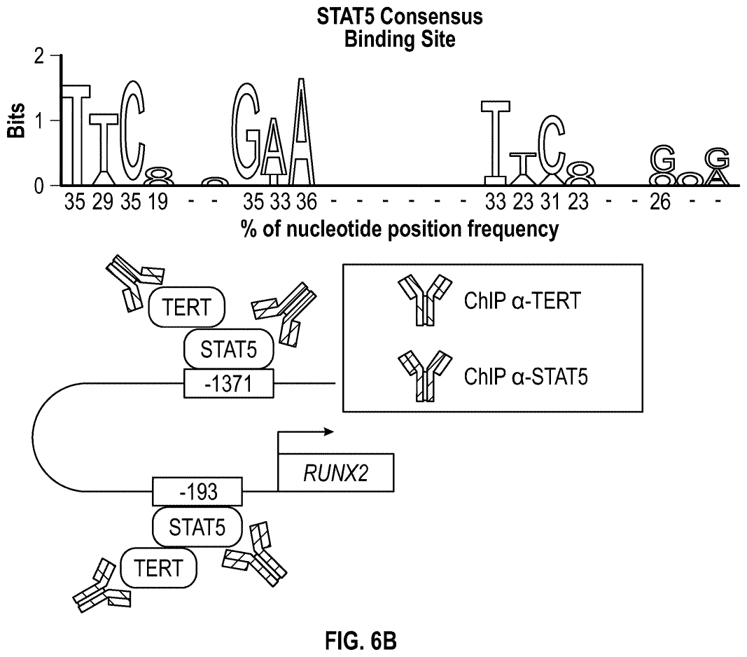

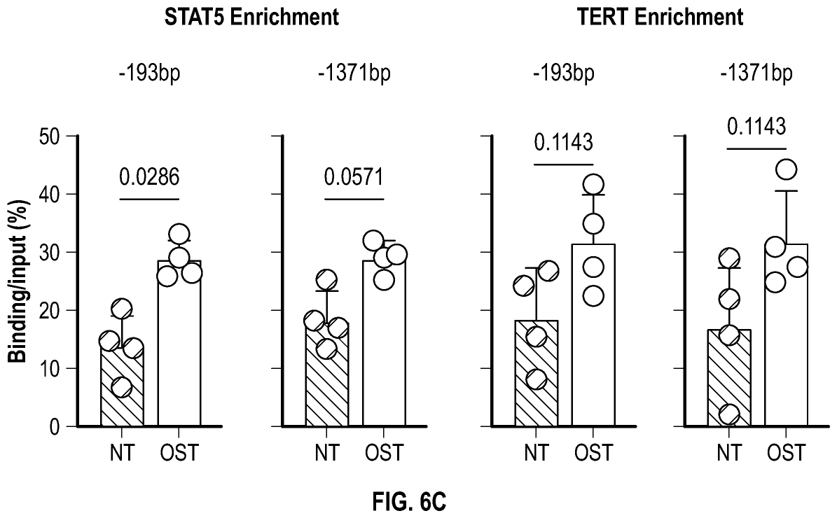

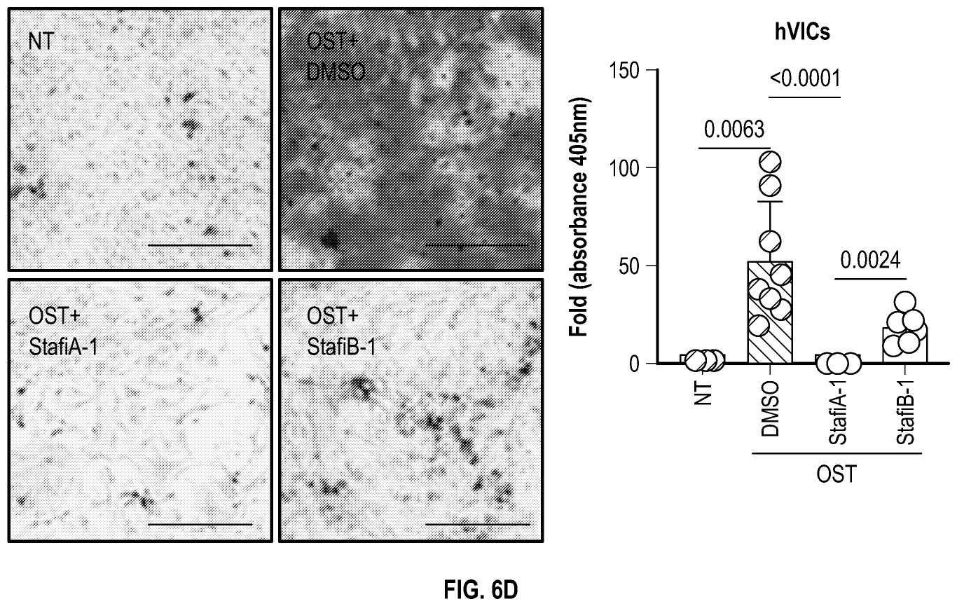

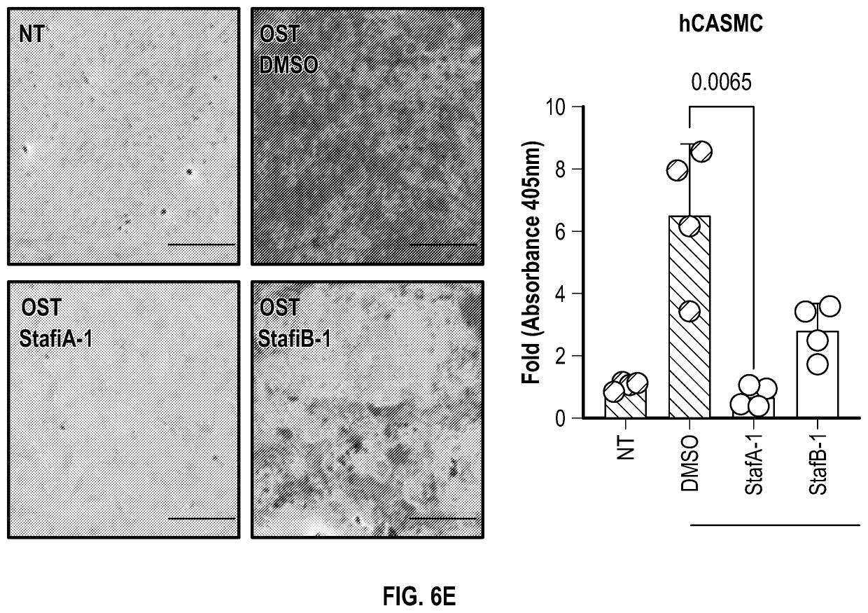

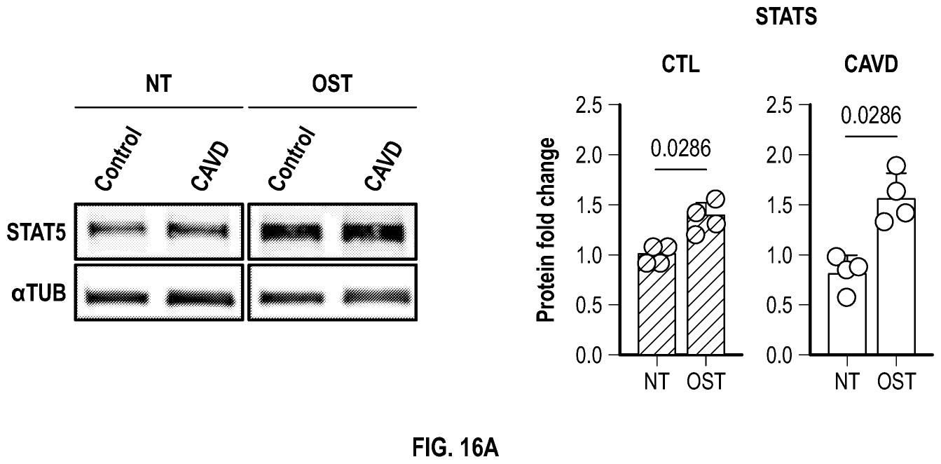

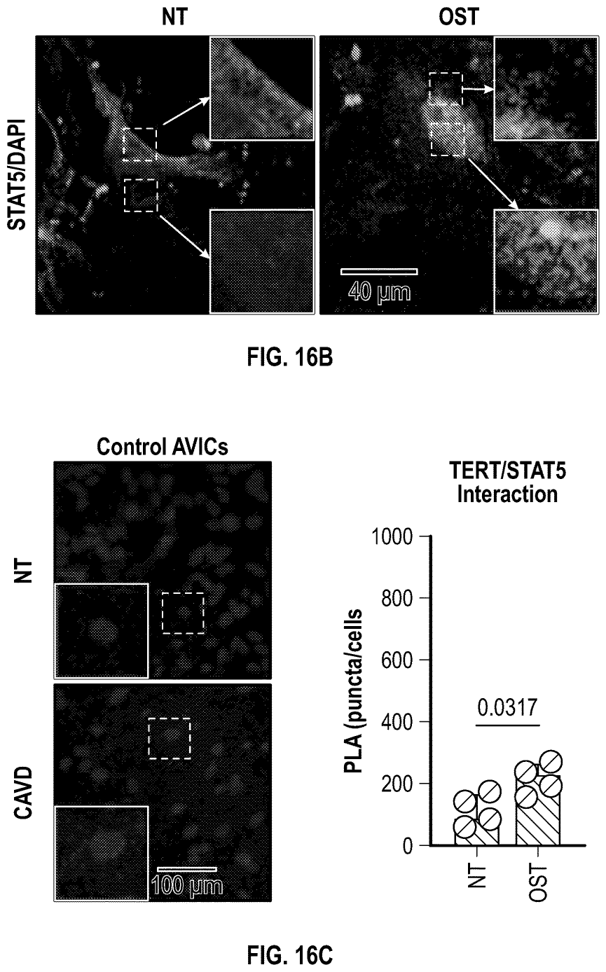

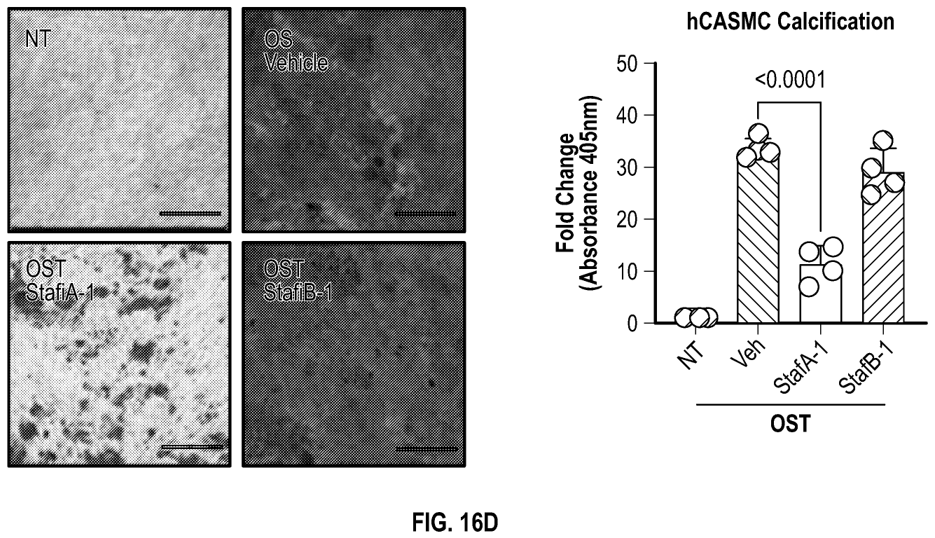

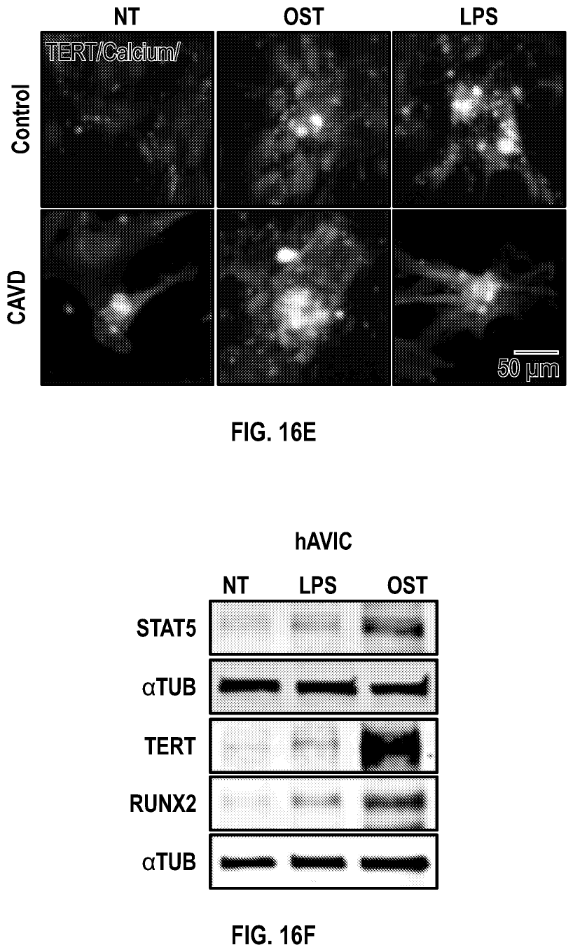

TERT exhibits transcriptional regulatory functions in various tissues and cells by physically interacting with transcription factors like NF-κB, Sp1, and E2F1. Of interest was STAT5A, STAT5A expression, but not STAT5B, was found to be upregulated during the osteogenic differentiation of AVICs (FIG. 3D). Therefore, the STAT5A role during the osteogenic differentiation of AVICs was assessed. OST stimulation was found to upregulate STAT5 protein in AVICs and STAT5 was detected in the nucleus of OST-stimulated AVICs (FIGS. 16A and 16B). Next, a proximity ligation assay (PLA) was used to examine TERT and STAT5 colocalization, as this technique is capable to detect interacting proteins in a proximity range below 40 nm. PLA revelated that TERT and STAT5 significantly colocalized in control AVICs after 21 days of osteogenic stimulation (FIG. 16C). Likewise, TERT/STAT5 colocalization was elevated in CAVD AVICs after 21 days of OST stimulation, at levels higher to those one observed for control AVICs (FIG. 6A). Together, these data strongly support a role for STAT5 during the osteogenic differentiation of AVICs.

Increased RUNX2 expression and activity is the hallmark of osteoblast differentiation and vascular calcification. RUNX2 is the master regulator of osteogenic differentiation and RUNX2 is downregulated in TERT-knockdown cells (FIG. 4). Among the non-canonical TERT functions, it has been described that TERT protein can interact with chromatin remodeling proteins and transcription factors to regulate gene expression. Thus, it was determined how RUNX2 expression is driven by TERT during osteogenic differentiation of AVICs. There was then a search for potential interacting partners by screening the RUNX2 promoter in the 5 kbp region upstream of the RUNX2 gene (NM_001015051.3) with LASAGNA and TRANSFAC/MATCH suites. Thirteen potential STAT5 binding sites were identified (Table 2). The sites −1371 bp and −193 bp, located in the upstream region of the RUNX2 promoter, both were highly similar to the tetrameric consensus STAT5 binding site, and show an elevated likelihood of being functional sites (core and matrix scores of 0.788 and 0.834, and 0.995 and 0.882, respectively, FIG. 6B). STAT5 and TERT binding to both −1371 bp and −193 bp sites at the promoter of RUNX2 were examined by chromatin immunoprecipitation (ChIP, FIG. 6B) as STAT5 binding to RUNX2 promoter has never been explored experimentally while it has been reported that TERT may act as a co-factor during transcription. ChIP assessments found that STAT5 binds to the RUNX2 promoter at both sites and that the binding enrichment was significantly higher during osteogenic differentiation (FIG. 6C). While it was also found that TERT also binds to −1371 bp and −193 bp of RUNX2 promoter, the trending enrichment during OST treatment did not reach statistical significance, showing that TERT itself does not bind to DNA directly but sits along to STAT5 on the RUNX2 gene promoter.

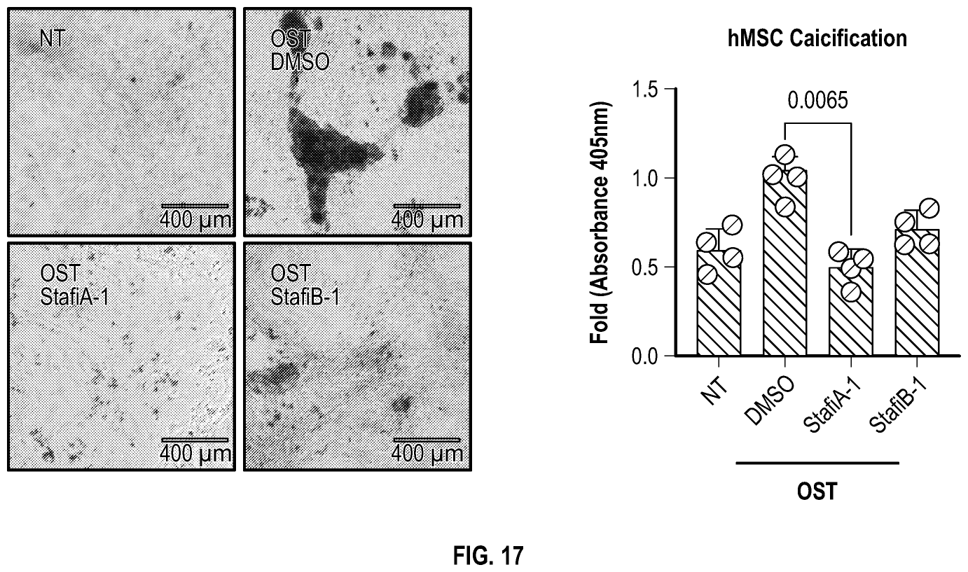

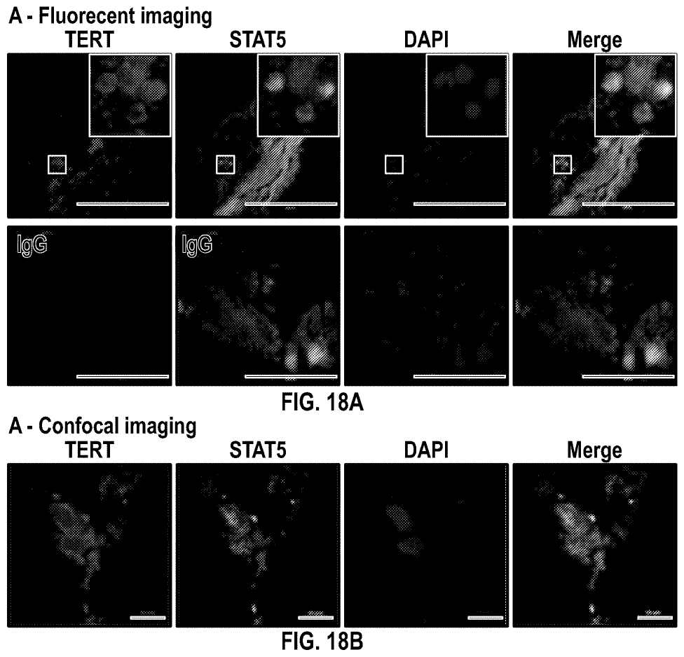

Two different genes encode STAT5 isoforms (STAT5A and STAT5B), but 96% of their amino acid sequence is shared while their transcriptional activities are redundant. Currently, there are no commercially available antibodies capable to distinguish between STAT5A and STAT5B isoforms. Thus, to assess the involvement of STAT5A and STAT5B isoforms in calcification, a pharmacological approach was utilized. Human A VICs were treated with the small molecules StafiA-1 and StafiB-1, which have been reported to specifically inhibit STAT5A and STAT5B, respectively. StafiA-1 significantly inhibited calcification of AVICs after 21 days of treatment and this inhibition was of a greater extent than the inhibition observed when AVICs were treated with StafiB-1, demonstrating that STAT5A is the main player during the osteogenic differentiation of AVICs (FIG. 6D). Similarly, StafiA-1 significantly reduced the calcification in hCASMC and hMSCs while StafiaB-1 did not (FIG. 16D and FIG. 17). These data show that TERT/STAT5A bind and activates the RUNX2 promoter during the osteogenic transition of AVICs.

Finally, the mechanism by which TERT and STAT5 become activated during the osteogenic differentiation of human AVICs was explored. As extensively reported, inflammation contributes to CAVD pathogenesis. Thus, bacterial lipopolysaccharide (LPS) was utilized to trigger inflammation in cultured human AVICs. AVICs were treated with daily doses of LPS alone and found that LPS was sufficient to induce calcification of AVICs. Like OST treatment, TERT-positive cells accumulated near calcified nodules after LPS and osteogenic stimulation (FIG. 16E). Protein analysis showed that LPS alone was also sufficient to upregulate TERT, RUNX2, and STAT5 proteins (FIG. 16F). Together, these observations show that LPS/TLR4 inflammatory signaling pathway participates in the regulation of TERT and the osteogenic transition of human AVICs.

### TERT and STAT5 are Upregulated in CAVD Tissue

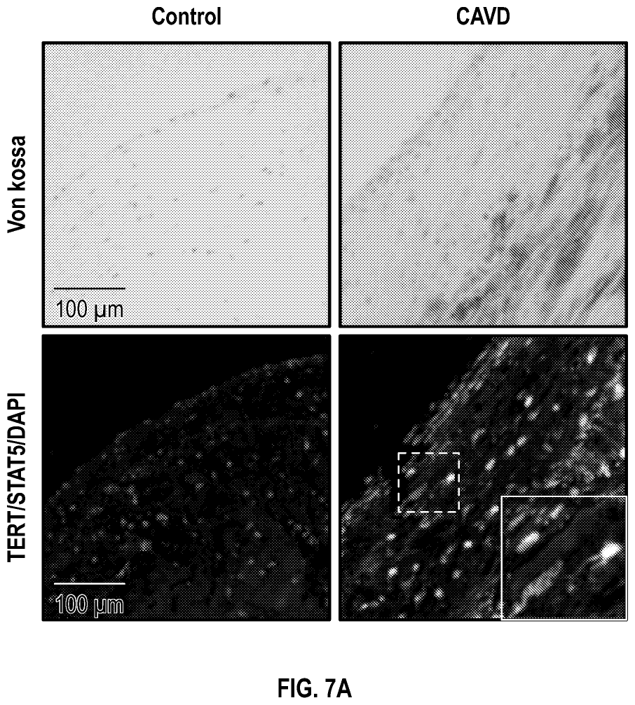

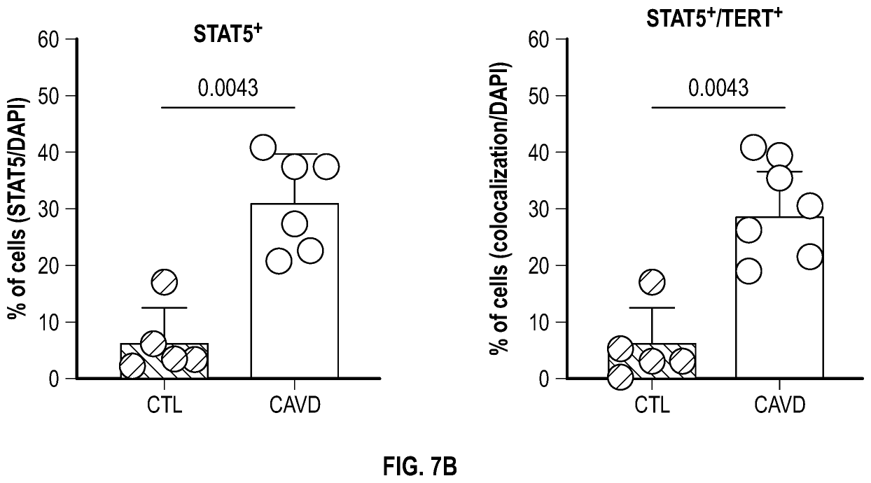

Having determined that STAT5A mediates TERT in calcification, STAT5 expression was assessed in human valves and found that STAT5 protein localized to areas of calcification, mirroring TERT distribution in the valve leaflet (FIG. 7A). Furthermore, the number of cell-positive for STAT5 was significantly increased in CAVD tissue and present in TERT-positive valve cells (FIG. 7B). Altogether, these data support in vitro investigations and show that TERT contributes to CAVD pathogenesis via its interaction with STAT5A.

### Example 2: Discussion

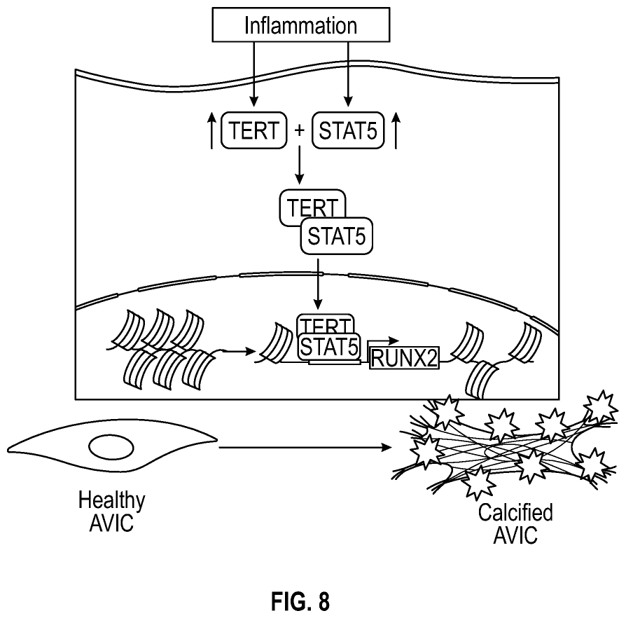

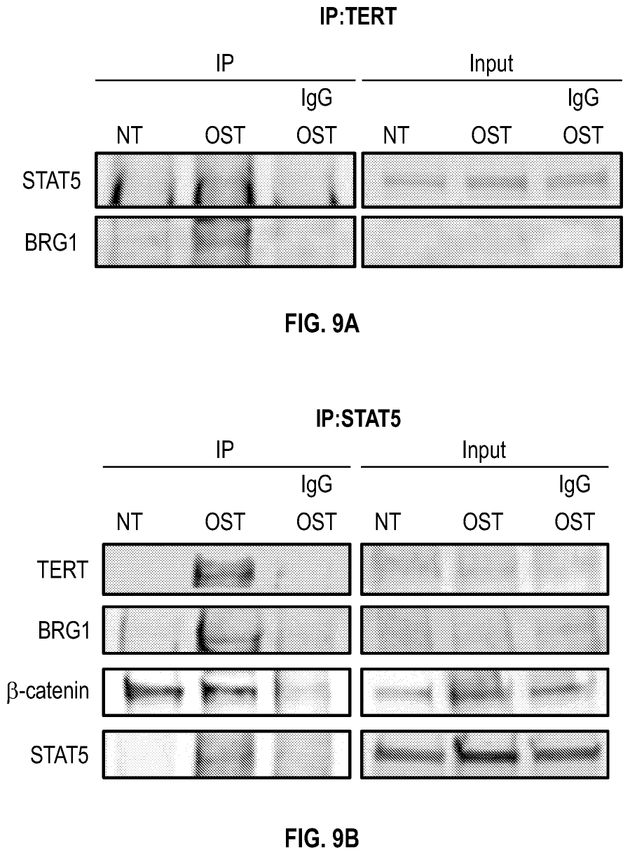

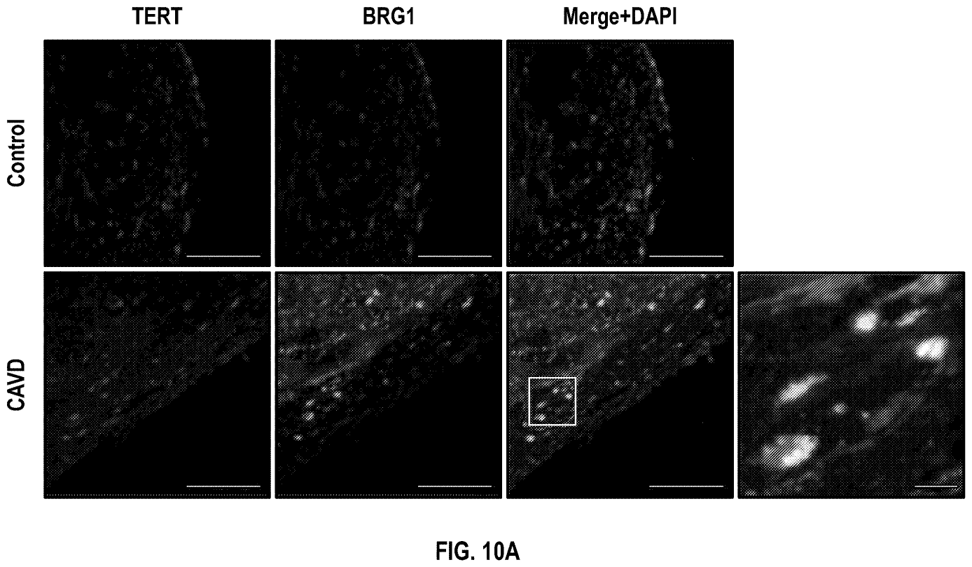

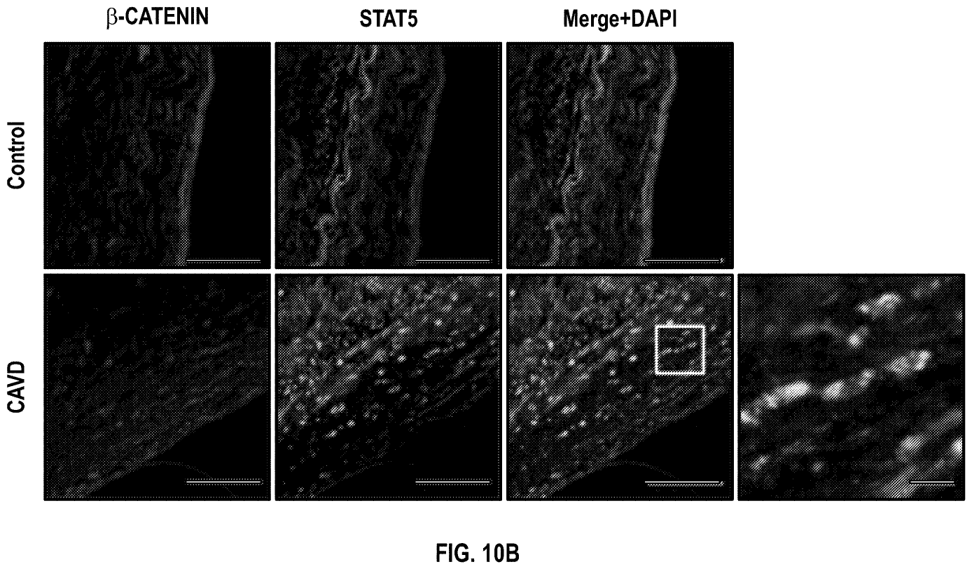

This study reveals a novel mechanism of TERT in driving a human disease pathology (FIG. 8). This disclosure establishes that TERT is upregulated in CAVD tissue and primary cell lines derived from those tissues, and that TERT is required for the osteogenic transition of vascular cells. The mechanism by which TERT promotes the osteogenic transition of AVICs is via interaction with STAT5, and this complex binds to the RUNX2 gene promoter. Finally, it was determined that TERT and STAT5 colocalized in CAVD tissue. Collectively, these experiments support a role for TERT in the osteogenic transition of AVICs and CAVD pathogenesis.

While TERT is well-known for its telomere-extending activity at the end of the chromosomes, it is now well recognized that TERT regulates gene expression and contributes to chromatin remodeling. Previous reports have shown that TERT physically interacts with the transcription factor NF-κB to promote the expression of several genes, including IL6, IL8, and TNF, cytokines critical for inflammation and cancer progression. Another study showed that TERT physically binds to the transcription factor Sp1 to activate the VEGF promoter to stimulate angiogenesis and vascular development. Further, it was shown that TERT participates in smooth muscle cell proliferation and neointima formation by interacting with the transcription factor E2F1 and stimulating its binding to S-phase genes. In addition to supporting transcription factor activity, TERT also interacts with the chromatin remodeling protein Brahma-related gene 1 (BRG1), a catalytic subunit of the mammalian SWI/SNF chromatin-remodeling BAF complex, where it serves as a chaperone to facilitate the recruitment of BRG1 and histone acetyltransferase activity to stimulate chromatin accessibility and transcription. It is shown that TERT interacts with STAT5 and together, are recruited to two consensus sites in the RUNX2 gene promoter during osteogenic differentiation of AVICs. Therefore, this shows that TERT serves as a transcription co-factor by enabling an accessible chromatin state amenable to STAT5 binding to the RUNX2 gene promoter. These findings that TERT is upregulated in CAVD tissue and during osteogenic differentiation. Furthermore, these observations that TERT depletion decreased RUNX2 expression and calcification, and the finding that TERT overexpression drives osteogenesis in MSC, support a role for TERT modulating cellular phenotypic transition and osteogenic reprogramming.

AVICs and SMCs are highly plastic and share calcification aspects of the transcriptional program observed during the differentiation of MSCs into osteoblasts, including the upregulation of RUNX2, BGLAP, TNAP, and the secretion of bone-forming proteins and accumulation of calcium minerals. In AVICs, mechanical stress, disturbed flow, and inflammation promote calcification as well as osteogenic differentiation. These observations show that AVICs phenotypically switch and undergo an osteogenic transition like MSCs, whereby AVICs acquire osteoblast-like characteristics. The mechanisms driving these cell transitions are not fully understood. It has been speculated that fully differentiated cells can de-differentiate into a transitory, multipotent stem cell-like state and then acquire an osteogenic phenotype. TERT was the focus as it is highly expressed in stem cells, overexpression of TERT drives osteogenesis in a MSC, and TERC deficiency does not alter the osteogenic capacity in a murine model of vascular calcification. It is shown that the upregulation of TERT leads healthy AVICs to undergo a de-differentiation process, likely to involve broad chromatin changes and that in an inflammatory milieu, TERT, and STAT5A couple to drive the osteogenic transition of AVICs.

The osteogenic differentiation of stem cells is orchestrated by RUNX2, which is also detected in calcified vascular cells. Another study showed that CAVD was diagnosed via ultrasound imaging, which detects leaflet mobility, however examining the macroscopic pictures and histological staining of valve leaflets used in this analysis shows that while these valves are indeed stiff, they do not exhibit robust nodules of calcification as do the valves in this data set (FIGS. 1A, 1C, 5, and 11A). Their analysis identified three VIC populations defined by expression of FOS, HSPA6, and SPARC. However, the cells from the six valves were pooled, with 10.4% of cells coming from control tissue and the remaining 89.6% from CAVD tissues. Bulk transcriptomic data from these tissues show only a 1.49-fold increase in RUNX2 gene expression in CAVD tissues. Inspection of publicly available bulk RNA-sequencing data (PRJNA552159) did not show any increase in TERT transcript expression. While hemodynamic data showed these valves had undergone fibrotic remodeling, only a low number of calcifying cells were present, hence, perhaps why TERT expression remained undetected in this approach. In the present disclosure a portion of each valve used was processed, and the calcification level was assessed by histological staining. Further, TERT expression was found to be dynamic, increasing sharply in AVICs under OST treatment then dropping (FIG. 14C) while elevated protein levels are sustained for longer (FIGS. 3D and 3E) and as observed in CAVD tissue (FIGS. 1C, 1D, and 6).

Cell replication progressively shortens telomeres, triggering senescence, yet telomere length varies widely between people of the same age. TL is considered a biomarker of aging, and several studies have utilized circulating leukocytes to investigate whether shortened telomeres correlate with various vascular disease states. One study correlated circulating leukocytes as surrogate to interrogate the TL from patients diagnosed calcific aortic stenosis (CAS) and found that CAS leukocytes had slightly shorter telomeres than age-matched healthy controls. However, telomere length in leukocytes is not indicative of global telomere length, telomerase complex activity, or TERT protein function in all cell types. First, short telomere may reflect increased circulating leukocyte turnover due to systemic inflammation, as leukocytes are highly proliferative. Second, telomere attrition might be a tissue- and disease-dependent process. Third, these correlative studies show no clear evidence of a causal link between telomere length, TERT activity, and disease progression. In this regard, the telomere length of the leukocytes of age-matched healthy controls and patients with atherosclerosis as well as the telomere length of the cells in the atherosclerotic plaques of the patients were compared in another study. They found that while telomere length in atherosclerosis leukocytes was significantly shorter than in control leukocytes, telomere length in the atherosclerotic plaques was significantly longer, indicative of increased telomerase complex activity. Therefore, leukocyte telomere length does not represent telomere length in the diseased tissue itself. The approach in the present disclosure examined TERT protein expression directly in the valve tissue and established patient specific AVIC lines for in vitro disease modeling. With these tools, it was determined that the expression levels of TERT and osteogenic markers in CAVD tissue were elevated, without changes in telomere length, proliferation, and DNA damage markers; thus, demonstrating a non-canonical role for TERT in these cells.

The function of STAT5 in bone formation is multifaceted. While it was shown that STAT5 can promote the differentiation of MSCs into osteoblasts by upregulation of the osteogenic genes in a Jak2-dependent manner, a Stat5a general knockout mouse exhibits elevated bone mass and bone mineral density, showing that STAT5A inhibition may enhance bone remodeling. These reports indicate that the role of STAT5 in bone formation and remodeling is complex and more studies are required. STAT5 has diverse functions: it interacts with histone acetyltransferases and transcription factors such as the glucocorticoid receptor, SP1, YY1, and C/EBPβ to stimulate gene expression, and two studies in cancer cells identified that STAT5 induces TERT expression. The present disclosure found multiple STAT5 binding sites in the RUNX2 promoter region and provide strong evidence in support of a mechanism in CAVD pathogenesis where TERT/STAT5 co-localize and translocate to the nucleus to activate osteogenic gene transcription which drives the early events in cellular osteogenic transition.

Current CAVD therapies are surgical, limited to either mechanical or bioprosthetic valve replacement, and performed only when the disease has progressed to the advanced point of affecting blood flow and heart function. The present disclosure has identified an innovative mechanism that contributes to CAVD pathogenesis by determining a causal link between TERT and the osteogenic differentiation of valve cells. This disclosure is the first to show the non-telomere extending function of TERT is operative in CAVD pathogenesis and has now uncovered a potential therapeutic target that could be leveraged for the pharmacological treatment of CAVD.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
Patient Information

| | | | Aortic Valve | | Calcification | | | | |
|---|---|---|---|---|---|---|---|---|---|
| | | | Leaflet | | (Von Kossa | | | | |
| Patient ID | Age | Sex | Number | Cause of Death | Staining) | Hypertension | Diabetes | Smoking | Alcohol |
| 2016-034 | 91 | M | 3 | Natural causes | Yes | No | No | No | Unknown |
| 2016-037 | 31 | M | 3 | Brain anoxia | No | No | No | Unknown | Yes |
| | | | | due to | | | | | |
| | | | | cardiovascular | | | | | |
| | | | | complications | | | | | |
| 2016-038 | 81 | M | 3 | Myocardial | No | No | No | Unknown | Unknown |
| | | | | Infarction | | | | | |
| 2016-042 | 22 | F | 3 | Brain anoxia | No | No | No | No | Unknown |
| | | | | due to head | | | | | |
| | | | | trauma | | | | | |
| 2016-045 | 59 | F | 3 | Stroke | No | PH | Yes | Yes | Unknown |
| 2016-046 | 65 | M | 3 | Head trauma | No | No | No | Unknown | Unknown |
| | | | | from accident | | | | | |
| 2016-048 | 56 | F | 3 | Stroke | No | HTN | No | Unknown | Unknown |
| 2016-050 | 65 | M | 3 | Stroke | No | HTN | No | Unknown | Yes |
| 2016-051 | 65 | M | 3 | Natural causes | Yes | HTN | No | Yes | Yes |
| 2016-053 | 73 | M | 3 | Congestive | Yes | No | No | No | Unknown |
| | | | | heart failure | | | | | |
| 2016-054 | 63 | F | 3 | Hemorrhage | No | No | Yes | No | Unknown |
| | | | | and Rupture | | | | | |
| | | | | AAA | | | | | |
| 2016-060 | 73 | M | 3 | Cardiac arrest | Yes | No | No | Unknown | Unknown |
| 2016-061 | 67 | M | 3 | Natural causes | Yes | No | Yes | Unknown | Unknown |
| 2016-062 | 59 | M | 3 | Cardiac arrest | Yes | No | No | Yes | Unknown |
| 2016-065 | 58 | M | 3 | Stroke | No | HTN | Yes | Yes | Unknown |
| 2016-068 | 68 | F | 3 | Intracranial | Yes | No | No | Yes | Unknown |
| | | | | hemorrhage | | | | | |
| 2016-072 | 62 | F | 3 | Cardiac arrest | Yes | No | No | No | Unknown |
| 2016-073 | 41 | F | 3 | Traumatic | No | HTN | No | Yes | Unknown |
| | | | | arrest and | | | | | |
| | | | | motor vehicle | | | | | |
| | | | | accident | | | | | |
| 2016-078 | 34 | F | 3 | Heart Failure | No | No | No | Unknown | Unknown |
| 2016-079 | 59 | M | 3 | Respiratory | No | HTN | No | No | Unknown |
| | | | | failure | | | | | |
| 2016-083 | 52 | M | 3 | Stroke | No | PAH | No | Yes | Unknown |
| 2016-085 | 52 | M | 3 | Myocardial | No | PH | No | Unknown | Unknown |
| | | | | Infarction | | | | | |
| 2016-091 | 59 | F | 3 | Stroke | No | HTN | No | Unknown | Unknown |
| 2016-092 | 47 | F | 3 | Stroke | No | No | No | No | Unknown |
| 2016-093 | 43 | M | 3 | Cardiac arrest | Yes | HTN | Yes | No | Unknown |
| 2016-095 | 87 | M | 3 | Myocardial | Yes | HTN | No | Yes | Unknown |
| | | | | Infarction | | | | | |
| 2016-098 | 57 | M | 3 | Cardiac arrest | No | HTN | No | Yes | Unknown |
| 2016-101 | 37 | F | 3 | Cardiac Death | No | No | No | Unknown | Unknown |
| 2016-102 | 58 | F | 3 | Cardiac arrest | Yes | No | No | Yes | Unknown |
| | | | | and asystole | | | | | |
| 2016-103 | 72 | M | 3 | Cardiac arrest | Yes | HIN | No | Unknown | Unknown |
| 2016-105 | 86 | M | 3 | Cardiac arrest | Yes | No | No | No | Unknown |
| 2016-118 | 62 | M | 3 | Cardiac arrest | Yes | No | No | No | Unknown |
| 2016-146 | 79 | M | 3 | Cardiac arrest | No | No | No | Yes | Unknown |
| 2017-005 | 66 | F | 3 | Stroke | No | No | No | Unknown | Unknown |
| 2017-025 | 57 | F | 3 | Brain anoxia | Yes | No | Yes | Unknown | Unknown |
| | | | | due to choking | | | Type II | | |
| 2017-035 | 48 | F | 3 | Brain anoxia | No | No | No | Unknown | Unknown |
| | | | | due to CO | | | | | |
| | | | | poisoning | | | | | |
| 2017-051 | 56 | M | 3 | Brain anoxia | No | NG | No | Unknown | Unknown |
| | | | | due to cardiac | | | | | |
| | | | | arrest | | | | | |
| 2017-069 | 37 | M | 3 | Motor vehicle | No | No | No | Unknown | Unknown |
| | | | | accident | | | | | |
| 2017-076 | 68 | F | 3 | Myocardial | Yes | No | No | No | Unknown |
| | | | | Infarction | | | | | |
| 03-0127 | 61 | M | 3 | Surgical | No | HTN | No | Unknown | Unknown |
| | | | | replacement | | | | | |
| 03-0129 | 68 | M | 3 | Surgical | No | HIN | Yes | Unknown | Unknown |
| | | | | replacement | | | Type II | | |

M = Male;
F = Female;
HTN = Hypertension;
PH = Pulmonary Hypertension;
PAH = Pulmonary Arterial Hypertension

TABLE 2
LASAGNA and TRANSFAC STAT5 Search on RUNX promoter

| Name | Position | DB | Strand |
|---|---|---|---|
| STAT5B (homodimer) | −4572 | TRANSFAC | (−) |
| STAT5A (homotetramer) | −2844 | TRANSFAC | (−) |
| STAT5A (homotetramer) | −1429 | LASAGNA | (−) |
| STAT5A (homotetramer) | −1372 | TRANSFAC | (−) |
| STAT5A | −1218 | LASAGNA | (+) |
| STAT5A | −430 | LASAGNA | (−) |
| STAT5A (homotetramer) | −279 | LASAGNA | (+) |
| STAT5A (homotetramer) | −194 | TRANSFAC | (+) |

TABLE 3

| Sequence | p-value |
|---|---|
| cttttccaagaaagc (SEQ ID NO: 4) | 0 |
| taccaacaactttttttctttgaaatt | 0.000325 |
| gatttcaagatc (SEQ ID NO: 5) | |
| tcctagttactgtcacactaggaa | 0.00015 |
| (SEQ ID NO: 6) | |
| taaatggcaaaaaaATGCCtagaa | 0.0005 |
| (SEQ ID NO: 7) | |
| gagttcta (SEQ ID NO: 8) | 0 |
| cagaattt (SEQ ID NO: 9) | 0.0004 |
| ttacaggagtttgggctccttcag | 0.000525 |
| (SEQ ID NO: 10) | |
| tgccaggaaaggccttaccacaag | 0 |
| (SEQ ID NO: 11) | |

TABLE 4
Primers

| | | Forward Primer Sequence | Reverse Primer Sequence | | |
|---|---|---|---|---|---|
| Species | Gene | 5′ -> 3′ | 5′ -> 3′ | Tech | Source |
| Human | TERT | GCATTGGAATCAGACAGCAC | CCACGACGTAGTCCATGTTC | qPCR | Sigma- |
| | | (SEQ ID NO: 12) | (SEQ ID NO: 13) | | Aldrich |
| Human | RUN | ATTCCTGTAGATCCGAGCAC | GCTCACGTCGCTCATTTTGC | qPCR | IDT |
| | X2 | C (SEQ ID NO: 14) | (SEQ ID NO: 15) | | |
| Human | MSX2 | GAGCTGGGATGTGGTAAAGG | AAATTCAGAAGATGGAGCG | qPCR | IDT |
| | | (SEQ ID NO: 16) | G (SEQ ID NO: 17) | | |
| Human | CDK | TGTCCGTCAGAACCCATG | AAAGTCGAAGTTCCATCGCT | qPCR | IDT |
| | N1A | (SEQ ID NO: 18) | C (SEQ ID NO: 19) | | |
| Human | PCNA | ACACTAAGGGCCGAAGATAA | ACAGCATCTCCAATATGGCT | qPCR | IDT |
| | | C (SEQ ID NO: 20) | GA (SEQ ID NO: 21) | | |
| Human | TP53 | GAGGTTGGCTCTGACTGTAC | TCCGTCCCAGTAGATTACC | qPCR | IDT |
| | | C (SEQ ID NO: 22) | AC (SEQ ID NO: 23) | | |
| Human | ALPL | ATGGGATGGGTGTCTCCACA | CCACGAAGGGGAACTTGTC | qPCR | IDT |
| | | (SEQ ID NO: 24) | (SEQ ID NO: 25) | | |
| Human | GLB1 | TATACTGGCTGGCTAGATCA | GGCAAAATTGGTCCCACCTA | qPCR | IDT |
| | | CTG (SEQ ID NO: 26) | TAA (SEQ ID NO: 27) | | |
| Human | STAT | CAGCAGCTTGACACACGGTA | GCCCAATCTTGACTCTCAAT | qPCR | IDT |
| | 3 | (SEQ ID NO: 28) | CC (SEQ ID NO: 29) | | |
| Human | STAT | ATGTGAAACCACAGATCAAG | TCTGTGGGTACATGTTATAG | qPCR | Sigma- |
| | 5A | (SEQ ID NO: 30) | G (SEQ ID NO: 31) | | Allrich |
| Human | STAT | ATGGGACTCAGTAGATCTTG | CTTCAGTAAAAACCCATCTT | qPCR | Sigma- |
| | 5B | (SEQ ID NO: 32) | CC (SEQ ID NO: 33) | | Aktrich |
| Human | TGFb | CTGCATCTGGTCACGGTCG | CCTCGGGCTCAGGATAGTCT | qPCR | IDT |
| | 2 | (SEQ ID NO: 34) | (SEQ ID NO: 35) | | |
| Human | OSX | CCCCCTTTACAAGCACTAATG | GGCAGACAGTCAGAAGAGC | qPCR | IDT |
| | | G (SEQ ID NO: 36) | TG (SEQ ID NO: 37) | | |
| Human | ACT | AAAAGACAGCTACGTGGGTG | GCCATGTTCTATCGGGTACT | qPCR | IDT |
| | A2 | A (SEQ ID NO: 38) | TC (SEQ ID NO: 39) | | |
| Human | FOX | TCGTCATAATCTGTCCCTACA | CGGCTTCGGCTCTTAGCAAA | qPCR | Sigma- |
| | 01 | CA (SEQ ID NO: 40) | (SEQ ID NO: 41) | | Aldrich |
| Human | 18s | GTAACCCGTTGAACCCCATT | CCATCCAATCGGTAGTAGCG | qPCR | IDT |
| | | (SEQ ID NO: 42) | (SEQ ID NO: 43) | | |
| Human | MYH | CGCCAAGAGACTCGTCTGG | TCTTTCCCAACCGTGACCTT | qPCR | IDT |
| | 11 | (SEQ ID NO: 44) | C (SEQ ID NO: 45) | | |
| Human | IL6 | AAATTCGGTACATCCTCGAC | GGAAGGTTCAGGTTGTTTTC | qPCR | IDT |
| | | GG (SEQ ID NO: 46) | TGC (SEQ ID NO: 47) | | |
| Human | VIM | GGAAACTAATCTGGATTCAC | CATCTCTAGTTTCAACCGTC | qPCR | Sigma- |
| | | TC (SEQ ID NO: 48) | (SEQ ID NO: 49) | | Aldrich |
| Human | SOX9 | GCCAGGTGCTCAAAGGCTA | TCTCGTTCAGAAGTCTCCAG | qPCR | IDT |
| | | (SEQ ID NO: 50) | AG (SEQ ID NO: 51) | | |
| Human | KLF4 | CCCACATGAAGCGACTTCCC | CAGGTCCAGGAGATCGTTGA | qPCR | IDT |
| | | (SEQ ID NO: 52) | A (SEQ ID NO: 53) | | |
| Human | SM22 | AGTGCAGTCCAAAATCGAGA | CTTGCTCAGAATCACGCCAT | qPCR | IDT |
| | | AG (SEQ ID NO: 54) | (SEQ ID NO: 55) | | |
| Human | TNF | CCTCTCTCTAATCAGCCCTCT | GAGGACCTGGGAGTAGATG | qPCR | IDT |
| | | G (SEQ ID NO: 56) | AG (SEQ ID NO: 57) | | |
| Human | BGL | GGACTGTGACGAGTTGGCTG | CCGTAGAAGCGCCGATAGG | qPCR | IDT |
| | AP | (SEQ ID NO: 58) | (SEQ ID NO: 59) | | |
| Human | SMT | CAAAAAGTCCTAACCCCTGC | TCATGTCGTCCACCTCCAC | qPCR | IDT |
| | N | T (SEQ ID NO: 60) | (SEQ ID NO: 61) | | |
| Human | BMP2 | ACCCGCTGTCTTCTAGCGT | TAAATTGAAGAAGAAGCGC | qPCR | Sigma- |
| | | (SEQ ID NO: 62) | C (SEQ ID NO: 63) | | Aldrich |
| Human | THBS | TGCTCCAATGCCACAGTTCC | CTGCTGAATTCCATTGCCAC | qPCR | IDT |
| | 1 | (SEQ ID NO: 64) | A (SEQ ID NO: 65) | | |
| Human | CNN1 | GTCAACCCAAAATTGGCACC | ACCTTGTTTCCTTTCGTCTTC | qPCR | IDT |
| | | A (SEQ ID NO: 66) | G (SEQ ID NO: 67) | | |
| Human | MMP | CACCAATTCCTGGGAAGTCT | GCAGCTGTTCACTTTGAGGA | qPCR | IDT |
| | 13 | (SEQ ID NO: 68) | (SEQ ID NO: 69) | | |
| Human | GAP | CTGGGCTACACTGAGCACC | AAGTGGTCGTTGAGGGCAAT | qPCR | IDT |
| | DH | (SEQ ID NO: 70) | G (SEQ ID NO: 71) | | |
| Human | pRU | ACCTTACAGGAGTTTGGGCT | CTTTCCTGGCATCCAGAAGG | ChIP | IDT |
| | NX2-1 | (SEQ ID NO: 72) | ATA (SEQ ID NO: 73) | | |
| Human | pRU | CCGCCCACCCCATTTACTT | GGCGAACAGACCAATTTTCT | ChIP | IDT |
| | NX2-2 | (SEQ ID NO: 74) | AGG (SEQ ID NO: 75) | | |
| Human | hTelomere | GGTTTTTGAGGGTGAGGGTG | TCCCGACTATCCCTATCCCT | PCR | Sigma- |
| | | AGGGT (SEQ ID NO: 76) | ATCCCT (SEQ ID NO: 77) | | Aldrich |

SEQUENCES
1.
SEQ ID NO: 1
AGWIQAQQLQGDALRQMQVLYGQHFPIEVRHYLAQWIESQPWDAIDLDNP
QDRAQATQLLEGLVQELQKKAEHQVGEDGFLLKIKLGHYATQLQKTYDRC
PLELVRCIRHILYNEQRLVREANNCSSPAGILVDAMSQKHLQINQTFEEL
RLVTQDTENELKKLQQTQEYFIIQYQESLRIQAQFAQLAQLSPQERLSRE
TALQQKQVSLEAWLQREAQTLQQYRVELAEKHQKTLQLLRKQQTIILDDE
LIQWKRRQQLAGNGGPPEGSLDVLQSWCEKLAEIIWQNRQQIRRAEHLCQ
QLPIPGPVEEMLAEVNATITDIISALVTSTFIIEKQPPQVLKTQTKFAAT
VRLLVGGKLNVHMNPPQVKATIISEQQAKSLLKNENTRNECSGEILNNCC
VMEYHQATGTLSAHFRNMSLKRIKRADRRGAESVTEEKFTVLFESQFSVG
SNELVFQVKTLSLPVVVIVHGSQDHNATATVLWDNAFAEPGRVPFAVPDK
VLWPQLCEALNMKFKAEVQSNRGLTKENLVFLAQKLFNNSSSHLEDYSGL
SVSWSQFNRENLPGWNYTFWQWFDGVMEVLKKHHKPHWNDGAILGFVNKQ
QAHDLLINKPDGTFLLRFSDSEIGGITIAWKFDSPERNLWNLKPFTTRDF
SIRSLADRLGDLSYLIYVFPDRPKDEVFSKYYTPVLAKAVDGYVKPQIKQ
VVPEFVNASADAGGSSATYMDQAPSPAVCPQAPYNMYPQNPDHVLDQDGE
FDLDETMDVARHVEELLRRPMDSLDSRLSPPAGLFTSARGSLS
2.
SEQ ID NO: 2
MAVWIQAQQLQGEALHQMQALYGQHFPIEVRHYLSQWIESQAWDSVDLDN
PQENIKATQLLEGLVQELQKKAEHQVGEDGFLLKIKLGHYATQLQNTYDR
CPMELVRCIRHILYNEQRLVREANNGSSPAGSLADAMSQKHLQINQTFEE
LRLVTQDTENELKKLQQTQEYFIIQYQESLRIQAQFGPLAQLSPQERLSR
ETALQQKQVSLEAWLQREAQTLQQYRVELAEKHQKTLQLLRKQQTIILDD
ELIQWKRRQQLAGNGGPPEGSLDVLQSWCEKLAEIIWQNRQQIRRAEHLC
QQLPIPGPVEEMLAEVNATITDIISALVTSTFIIEKQPPQVLKTQTKFAA
TVRLLVGGKLNVHMNPPQVKATIISEQQAKSLLKNENTRNDYSGEILNNC
CVMEYHQATGTLSAHFRNMSLKRIKRSDRRGAESVTEEKFTILFESQFSV
GGNELVFQVKTLSLPVVVIVHGSQDNNATATVLWDNAFAEPGRVPFAVPD
KVLWPQLCEALNMKFKAEVQSNRGLTKENLVFLAQKLFNNSSSHLEDYSG
LSVSWSQFNRENLPGRNYTFWQWFDGVMEVLKKHLKPHWNDGAILGFVNK
QQAHDLLINKPDGTFLLRFSDSEIGGITIAWKFDSQERMFWNLMPFTTRD
FSIRSLADRLGDLNYLIYVFPDRPKDEVYSKYYTPVPCESATAKAVDGYV
KPQIKQVVPEFVNASADAGGGSATYMDQAPSPAVCPQAHYNMYPQNPDSV
LDTDGDFDLEDTMDVARRVEELLGRPMDSQWIPHAQS
3.
SEQ ID NO: 3
MPRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAFG
FALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCE
RAWNHSVREAGVPLGLPAPGARRRGGSASRSLPLPKRPRRGAAPEPERTP
VGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSL
RPSLTGARRLVETIFLGSRPWMPGTPRRLPRLPQRYWQMRPLFLELLGNH
AQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKH
AKLSLQELTWKMSVRDCAWLRRSPGVGCVPAAEHRLREEILAKFLHWLMS
VYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKR
AERLTSRVKALFSVLNYERARRPGLLGASVLGLDDIHRAWRTFVLRVRAQ
DPPPELYFVKVDVTGAYDTIPQDRLTEVIASIIKPQNTYCVRRYAVVQKA
AHGHVRKAFKSHVSTLTDLQPYMRQFVAHLQETSPLRDAVVIEQSSSLNE
ASSGLFDVFLRFMCHHAVRIRGKSYVQCQGIPQGSILSTLLCSLCYGDME
NKLFAGIRRDGLLLRLVDDFLLVTPHLTHAKTFLRTLVRGVPEYGCVVNL
RKTVVNFPVEDEALGGTAFVQMPAHGLFPWCGLLLDTRTLEVQSDYSSYA
RTSIRASLTFNRGFKAGRNMRRKLFGVLRLKCHSLFLDLQVNSLQTVCTN
IYKILLLQAYRFHACVLQLPFHQQVWKNPTFFLRVISDTASLCYSILKAK
NAGMSLGAKGAAGPLPSEAVQWLCHQAFLLKLTRHRVTYVPLLGSLRTAQ
TQLSRKLPGTTLTALEAAANPALPSDFKTILD
4.
SEQ ID NO: 4
CTTTTCCAAGAAAGC
5.
SEQ ID NO: 5
TACCAACAACTTTTTTTCTTTGAAATTGATTTCAAGATC
6.
SEQ ID NO: 6
TCCTAGTTACTGTCACACTAGGAA
7.
SEQ ID NO: 7
TAAATGGCAAAAAAatgccTAGAA
8.
SEQ ID NO: 8
GAGTTCTA
9.
SEQ ID NO: 9
CAGAATTT
10.
SEQ ID NO: 10
TTACAGGAGTTTGGGCTCCTTCAG
11.
SEQ ID NO: 11
TGCCAGGAAAGGCCTTACCACAAG
12.
SEQ ID NO: 12
GCATTGGAATCAGACAGCAC
13.
SEQ ID NO: 13
CCACGACGTAGTCCATGTTC
14.
SEQ ID NO: 14
ATTCCTGTAGATCCGAGCACC
15.
SEQ ID NO: 15
GCTCACGTCGCTCATTTTGC
16.
SEQ ID NO: 16
GAGCTGGGATGTGGTAAAGG
17.
SEQ ID NO: 17
AAATTCAGAAGATGGAGCGG
18.
SEQ ID NO: 18
TGTCCGTCAGAACCCATG
19.
SEQ ID NO: 19
AAAGTCGAAGTTCCATCGCTC
20.
SEQ ID NO: 20
ACACTAAGGGCCGAAGATAAC
21.
SEQ ID NO: 21
ACAGCATCTCCAATATGGCTGA
22.
SEQ ID NO: 22
GAGGTTGGCTCTGACTGTACC
23.
SEQ ID NO: 23
TCCGTCCCAGTAGATTACCAC
24.
SEQ ID NO: 24
ATGGGATGGGTGTCTCCACA
25.
SEQ ID NO: 25
CCACGAAGGGGAACTTGTC
26.
SEQ ID NO: 26
TATACTGGCTGGCTAGATCACTG
27.
SEQ ID NO: 27
GGCAAAATTGGTCCCACCTATAA
28.
SEQ ID NO: 28
CAGCAGCTTGACACACGGTA
29.
SEQ ID NO: 29
GCCCAATCTTGACTCTCAATCC
30.
SEQ ID NO: 30
ATGTGAAACCACAGATCAAG
31.
SEQ ID NO: 31
TCTGTGGGTACATGTTATAGG
32.
SEQ ID NO: 32
ATGGGACTCAGTAGATCTTG
33.
SEQ ID NO: 33
CTTCAGTAAAAACCCATCTTCC
34.
SEQ ID NO: 34
CTGCATCTGGTCACGGTCG
35.
SEQ ID NO: 35
CCTCGGGCTCAGGATAGTCT
36.
SEQ ID NO: 36
CCCCCTTTACAAGCACTAATGG
37.
SEQ ID NO: 37
GGCAGACAGTCAGAAGAGCTG
38.
SEQ ID NO: 38
AAAAGACAGCTACGTGGGTGA
39.
SEQ ID NO: 39
GCCATGTTCTATCGGGTACTTC
40.
SEQ ID NO: 40
TCGTCATAATCTGTCCCTACACA
41.
SEQ ID NO: 41
CGGCTTCGGCTCTTAGCAAA
42.
SEQ ID NO: 42
GTAACCCGTTGAACCCCATT
43.
SEQ ID NO: 43
CCATCCAATCGGTAGTAGCG
44.
SEQ ID NO: 44
CGCCAAGAGACTCGTCTGG
45.
SEQ ID NO: 45
TCTTTCCCAACCGTGACCTTC
46.
SEQ ID NO: 46
AAATTCGGTACATCCTCGACGG
47.
SEQ ID NO: 47
GGAAGGTTCAGGTTGTTTTCTGC
48.
SEQ ID NO: 48
GGAAACTAATCTGGATTCACTC
49.
SEQ ID NO: 49
CATCTCTAGTTTCAACCGTC
50.
SEQ ID NO: 50
GCCAGGTGCTCAAAGGCTA
51.
SEQ ID NO: 51
TCTCGTTCAGAAGTCTCCAGAG
52.
SEQ ID NO: 52
CCCACATGAAGCGACTTCCC
53.
SEQ ID NO: 53
CAGGTCCAGGAGATCGTTGAA
54.
SEQ ID NO: 54
AGTGCAGTCCAAAATCGAGAAG
55.
SEQ ID NO: 55
CTTGCTCAGAATCACGCCAT
56.
SEQ ID NO: 56
CCTCTCTCTAATCAGCCCTCTG
57.
SEQ ID NO: 57
GAGGACCTGGGAGTAGATGAG
58.
SEQ ID NO: 58
GGACTGTGACGAGTTGGCTG
59.
SEQ ID NO: 59
CCGTAGAAGCGCCGATAGG
60.
SEQ ID NO: 60
CAAAAAGTCCTAACCCCTGCT
61.
SEQ ID NO: 61
TCATGTCGTCCACCTCCAC
62.
SEQ ID NO: 62
ACCCGCTGTCTTCTAGCGT
63.
SEQ ID NO: 63
TAAATTGAAGAAGAAGCGCC
64.
SEQ ID NO: 64
TGCTCCAATGCCACAGTTCC
65.
SEQ ID NO: 65
CTGCTGAATTCCATTGCCACA
66.
SEQ ID NO: 66
GTCAACCCAAAATTGGCACCA
67.
SEQ ID NO: 67
ACCTTGTTTCCTTTCGTCTTCG
68.
SEQ ID NO: 68
CACCAATTCCTGGGAAGTCT
69.
SEQ ID NO: 69
GCAGCTGTTCACTTTGAGGA
70.
SEQ ID NO: 70
CTGGGCTACACTGAGCACC
71.
SEQ ID NO: 71
AAGTGGTCGTTGAGGGCAATG
72.
SEQ ID NO: 72
ACCTTACAGGAGTTTGGGCT
73.
SEQ ID NO: 73
CTTTCCTGGCATCCAGAAGGATA
74.
SEQ ID NO: 74
CCGCCCACCCCATTTACTT
75.
SEQ ID NO: 75
GGCGAACAGACCAATTTTCTAGG
76.
SEQ ID NO: 76
GGTTTTTGAGGGTGAGGGTGAGGGT
77.
SEQ ID NO: 77
TCCCGACTATCCCTATCCCTATCCCT

1. A method of treating calcification of a circulatory system member in a subject comprising administering to the subject a pharmaceutically effective amount of a STAT5 inhibitor and/or a TERT inhibitor.

2. The method of claim 1, wherein the circulatory system member is a heart valve.

3. The method of claim 1, wherein the circulatory system member is a heart tissue.

4. The method of claim 1, wherein the circulatory system member is a heart vein or a heart artery.

5. The method of claim 1, wherein the subject has a calcific aortic heart disease, atherosclerotic plaques in an artery, or medial arterial calcification.

6. The method of claim 1, wherein the STAT5 inhibitor is administered to a vein of the subject.

7. (canceled)

8. (canceled)

9. (canceled)

10. A method of reducing calcification of a device within a subject's circulatory system comprising applying to the device a pharmaceutically effective amount of a STAT5 inhibitor and/or a TERT inhibitor.

11. The method of claim 10, wherein the device is a bioprosthetic heart valve.

12. The method of claim 10, wherein the device is a stent.

13. (canceled)

14. The method of claim 1, wherein the STAT5 inhibitor comprises a compound of Formula I, or a pharmaceutically acceptable salt or derivative thereof.

15. The method of claim 1, wherein the STAT5 inhibitor comprises a compound of Formula IA, or a pharmaceutically acceptable salt or derivative thereof.

16. (canceled)

17. (canceled)

18. (canceled)

19. The method claim 1, wherein the STAT5 inhibitor is selected from the group consisting of a StafiA-1, a StafiB-1, a pimozide, a nicotinoyl hydrazine, a BP-1-108, an AC-4-130, and a SF-1-088.

20. (canceled)

21. The method claim 1, wherein the STAT5 is a STAT5A.

22. The method claim 1, wherein the STAT5 is a STAT5B.

23. A method of identifying one or more small molecule inhibitors to reduce calcification, the method comprising:
introducing a STAT5 polypeptide and a TERT polypeptide in the presence of the one or more small molecule inhibitors,
identifying the one or more small molecule inhibitors that reduce or prevent interaction between the STAT5 and TERT polypeptides, and
wherein the one or more small molecule inhibitors reduce calcification of a circulatory system member or calcification of a device within a subject's circulatory system.

24. (canceled)

25. (canceled)

26. (canceled)

27. (canceled)

28. (canceled)

29. The method of claim 1, wherein the subject is an animal or a human.

30. A device comprising a pharmaceutically effective amount of a STAT5 inhibitor and/or a TERT inhibitor.

31. The device of claim 30, wherein the STAT5 inhibitor comprises a compound of Formula I, or a pharmaceutically acceptable salt or derivative thereof.

32. The device of claim 30, wherein the STAT5 inhibitor comprises a compound of Formula IA, or a pharmaceutically acceptable salt or derivative thereof.

33. The device of claim 30, wherein the STAT5 inhibitor comprises a StafiA-1, a StafiB-1, or a derivative thereof.

34. The device of claim 30, wherein the STAT5 inhibitor comprises a StafiA-1.

35. The device of claim 30, wherein the STAT5 inhibitor comprises a StafiB-1.

36. The device of claim 30, wherein the device is a stent or a prosthetic heart valve.
